(19) United States
(12) Patent Application Publication
(10) Pub. No.: US 2006/0078955 A1
(43) Pub. Date: Apr. 13, 2006

(54) Method for retrieving delta9-THC from oral fluid

(75) Inventors: Cheng-I Lin, Cupertino, CA (US); Tom Chia, Fountain Valley, CA (US); Marie Lin, Cupertino, CA (US); Michael J. Coyer, Dumore, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Lin-Zhi International, Sunnyvale, CA (US)

(21) Appl. No.: 10/965,067

(22) Filed: Oct. 13, 2004

(51) Int. Cl. G01N 33/543; G01N 33/537

(52) U.S. Cl. 435/7.92

(57) ABSTRACT

The present invention discloses methods and kits for retrieving a cannabinoid from an oral fluid collection device having adsorbed the cannabinoid and to prepare an oral fluid specimen for the determination of Δ⁹-tetrahydrocaninabinol (Δ⁹-THC) concentration qualitatively or quantitatively by an enzyme immunoassay. The methods and kits are particularly useful in preserving the Δ⁹-THC quantitatively from oral fluid samples during the process of collection and preparation.

### FIELD OF THE INVENTION

The present invention relates to the field of immunoassays. The invention provides methods for determining the amount of a cannabinoid, especially, Δ⁹-tetrahydrocannabinol (THC), in an oral fluid specimen suspected of containing the cannabinoid. In particular, the invention relates to methods and kits for retrieving a cannabinoid from an oral fluid collection device having adsorbed the cannabinoid.

### BACKGROUND OF THE INVENTION

Due to the wide-spread use of controlled substances or narcotics such as cannabinoids (marijuana), cocaine, amphetamines, and the like, it has become desirable to institute drug testing of athletes and others who are engaged in an occupation involving a public trust or in which an injury can occur if the person is not completely alert. Narcotic screening has become extensive practice in industry, business, the Armed Forces, schools and in the courts and prison systems. Such screening is used both as a pre-employment procedure and as a monitoring tool. There are a number of occasions on which a sample, particularly a sample of a biological fluid obtained from an individual, is to be screened for the presence of one or more cannabinoids. For instance, samples of blood, urine, or other biological fluids from applicants for certain permits or licenses may be checked for the presence of cannabinoids. Samples of individuals undergoing a drug treatment program may be screened for the presence of drugs, such as cannabinoids. In particular, the measurement of trace amounts of cannabinoids has become essential for many health care applications in pharmaceutical studies, therapeutic drug monitoring, and for drug abuse detection. Thus, there is a continuing interest in developing new, simpler more rapid and more sensitive techniques to measure the presence of cannabinoids in samples suspected of containing a cannabinoid.

Naturally occurring cannabinoids may be divided into two categories, endogenous and plant-derived. Endogenous cannabinoids (endocannabinoids) are a class of lipid-like molecules that share receptor binding sites with plant-derived cannabinoids and mimic many of their neurobehavioral effects (Mechhoulam et al., Adv. Exp. Bio. Med. (1996) 402:95-101). Two endocannabinoids have been characterized in some detail: arachidonyl ethanolamide (anandamide; Devane et al., Science (1992) 258:1946-1949) and 2-arachidonyl glyceride (2-AG; Mechoulam et al., Biochem. Pharmacol. (1995) 50:83-90). Like plant-derived cannabinoids, both anandamine and 2-AG are capable of eliciting hypotension (Varga et al., FASEB J. (1998) 12:1035-1044).

Plant-derived cannabinoids are known to elicit dramatic psychobehavioral effects and are also known to have complex cardiovascular effects, a prominent component of which is hypotension and bradycardia induced by e.g., Δ⁹-tetrahydrocannabinol (Vollmer et al., J. Pharm. Pharmacol. (1974) 26(3):186-192). Δ⁹-tetrahydrocannabinol (THC) and, to a small extent, Δ⁸-THC are the biologically active constituents in extracts of the plant Cannabis sativa (marijuana, hashish).

Marijuana based medicines have been a mainstay of many folk, herbal remedies and many beneficial pharmacological properties can be attributed to marijuana. Among them are analgesia, anti-inflammation, immunosuppression, anticonvulsion, lowering blood pressure and alleviation of intraocular pressure in glaucoma, anti-emetic activity, e.g., attenuation of vomiting (Munro et al., Nature (1993) 365:61-65; Hollister, Pharmacol. Rev. (1986) 38:1-20; Dewley, Pharmacol. Rev. (1986) 38:151-178). Other applications include appetite stimulation and treatment of cramps, migraines, pain, AIDS-related weight loss or “wasting,” muscle spasms in multiple sclerosis as well as other problems. However, also negative effects of marijuana use have been well documented. The negative pharmacological effects associated with marijuana (and shown to be associated with Δ⁹-THC) include psychological distortions of perception, loss of short-term memory, loss of motor coordination, sedation, and euphoria. Cannabinoids also increase heart rate and vary systemic arterial pressure.

THC is on the market as Marinol® (generic name dronabinol) and is used as an appetite stimulant, in the treatment of AIDS-related anorexia and for the control of nausea and vomiting associated with cancer chemotherapy. Nabilone®, a synthetic cannabinoid has been reported to be an anti-emetic and anxiolytic, and also useful for treating pain of various etiologies such as multiple sclerosis (MS), peripheral neuropathy and spinal injuries (Martyn et al., Lancet (1995) 345:579).

Because of its psychoactive properties, marijuana became a popular recreational drug of abuse and the major active ingredient, the cannabinoid Δ⁹-THC, has been used as a psychoactive agent for thousands of years. Long term use of marijuana is considered by many to lead to addiction. There is much debate over whether marijuana use should be legalized in certain cases, such as its use in cancer patients for ameliorating the nausea induced by chemotherapy or to lower intra-ocular pressure in glaucoma patients. The controversy regarding the medicinal use of marijuana is centered not only on what is delivered but on how it is delivered. Specifically, the primary method used to deliver marijuana into a patient's system is by smoking the marijuana; however, smoking increases an individual's risk for cancer, lung damage and emphysema. Furthermore, as discussed above, marijuana does contain high levels of the psychoactive drug, Δ⁹-THC. As such, there has been considerable debate as to whether or not the potential health benefits of smoking marijuana outweigh the health benefits.

However, it is of note that Δ⁹-THC is only one of a family of about 60 lipophylic bi- and tri-cyclic compounds named cannabinoids. These compounds usually contain a 1,1′-di-methyl-pyrane ring, a variedly derivatized aromatic ring and a variedly unsaturated cyclohexyl ring, and include for example the non-psychoactive cannabinol, cannabidiol and cannbinolic acid. These latter compounds have been suggested to contribute to some of the beneficial effects of Cannabis, such as cell protection, immunosuppression and anti-inflammatory properties suggesting that these non-psychoactive cannabinoids recognize the same cellular receptors as Δ⁹-THC, but, due to structural differences, do not have the same side effects.

After the elucidation of the THC structure, several synthetic compounds were prepared and found to be effective for the treatment of pain in cancer patients. Among these are: nabilone, naboctate and levonantradol. Although these drugs are useful, they have to a greater or lesser extent some of the negative pharmacological properties of THC.

Cannabinoids exert their effects by binding to specific receptors located in the cell membrane. Two cannabinoid receptors have been described, the CB1 receptor expressed primarily in the brain (Matsuda et al., Nature (1990) 346:561-564), and CB2 receptors expressed by cells of the immune system (Munro et al., Nature (1993) 365:61-65). CB1 receptor has been implicated in cannabinoid-induced hypotension and bradycardia (Varga et al., Eur. J. Pharmacol. (1995) 278:279-283).

The screening for a cannabinoid is typically done to determine whether the cannabinoid in question is present in the body (that is, in a sample of biological fluid) of the individual in question. Typically, the screening is to be conducted not only to determine whether detectable amounts of the cannabinoids in question are present in the sample, but whether a particular substance is present in an amount greater than a predetermined level. Such a level is also known as a “cutoff level.” These levels may be set by an organizational rule, e.g. an employer's rule, or by a law, for example, a maximum level of blood alcohol for one driving a vehicle, or a maximum amount of a steroid or other performance-enhancing substance for one to compete in an athletic event.

Increase in the use of marijuana have led to the development of assays for the detection of the primary active constituent, Δ⁹-THC, and more particularly, metabolites of Δ⁹-THC in urine and blood samples. The most common commercial assays employ the use of labeled cannabinoid derivatives in conjunction with antibodies against metabolites of the drug.

In practice, a urine or blood sample suspected of containing Δ⁹-THC metabolites (including glucuronides and other conjugation products) is contacted with antibodies in the presence of a labeled cannabinoid derivative. To the extent that Δ⁹-THC metabolites are present in the sample, there will be competition for binding to the antibodies, and the amount of the labeled cannabinoid derivative that remains bound will be reduced in proportion to the degree of competition with Δ⁹-THC metabolite in the sample. In other words, the extent to which the Δ⁹-THC metabolites in the sample can inhibit the binding of the labeled cannabinoid derivative to the antibody is a direct measurement of the amount of the Δ⁹-THC metabolite present in the sample. U.S. patent application Ser. No. 10/163,018 (Publication No. US-2003-0224373-A1) and Ser. No. 10/927,823, the disclosures of which are incorporated hereby in their entirety, describe homogenous enzyme immunoassays for the simultaneous detection of multiple analytes and for measuring an analyte in an oral fluid sample, respectively.

Because of higher cannabinoid concentration in urine, blood, serum or plasma samples, these bodily fluids represented the samples of choice for cannabinoid measurements. However, privacy concerns of individuals whose samples will be analyzed coupled with the desire or need to visually control collection of the test sample and considering health issues (HIV, hepatitis, etc.) involved in collecting blood, serum or plasma samples often make the collection of these samples impractical. Thus, it would be much more desirable to collect and analyze oral fluid samples instead of urine, blood, serum or plasma samples. Instead of spitting oral fluid into a container, collection devices are designed to allow an individual placing the device into the mouth to adsorb the oral fluid for convenience and ease of use.

Oral fluid has recently been widely used as a specimen in pharmaceutical studies, therapeutic drug monitoring, and for detection of drug abuse. The currently available oral fluid testing methods include conventional ELISA and on-site dip-stick testing, which are time consuming, labor intensive, costly, and of low precision.

Despite the widespread use of immunoassays, there are still difficulties in the measurement of cannabinoids derived from particular sample types, notably oral fluids, in which the cannabinoid may be present at very low concentrations. Thus, while the techniques described in U.S. Pat. Nos. 3,817,837, 6,033,890, 6,090,567, and 6,455,288 and U.S. patent application Ser. Nos. 10/163,018 and 10/927,823 substantially improved the efficiency of testing various analytes, including cannabinoids, they do not resolve the problem of measuring the presence of a cannabinoid in an oral fluid sample of low cannabinoid concentration. The main difficulty of applying the homogeneous enzyme immunoassay, described in U.S. patent application Ser. Nos. 10/163,018 and 10/927,823, to oral fluid is due to the low cannabinoid concentration in the oral fluid specimen and due to the stickiness, i.e., adherence or adsorption, of the cannabinoid to an oral fluid collection device.

For example, guidelines provided by the Substance Abuse and Mental Health Services Administration (SAMHSA; Federal Register, 2004; 69(71), 19673-19719) show that the detection limit, i.e., the recommended cutoff levels for various analytes in oral fluid samples are much lower than those for urine samples:

| Drug Category | Urine | | Oral Fluid (OF) | | Treated OF | |
|---|---|---|---|---|---|---|
| Amphetamines | 1000 | ng/mL | 50 | ng/ml | 12.5-25 | ng/ml |
| Cocaine metabolite | 150 | ng/mL | 20 | ng/ml | 5-10 | ng/ml |
| Methamphetamine | 500 | ng/ml | 50 | ng/ml | 12.5-25 | ng/ml |
| Ecstasy (MDMA) | 300 | ng/ml | 50 | ng/ml | 12.5-25 | ng/ml |
| Methadone | 300 | ng/ml | 40 | ng/ml | 10-20 | ng/ml |
| Opiate | 2000 | ng/mL | 40 | ng/ml | 10-20 | ng/ml |
| Phencyclidine | 25 | ng/mL | 10 | ng/ml | 2.5-5 | ng/ml |
| THC | 50 | ng/mL | 4 | ng/ml | 1-2 | ng/ml |

The low cutoff concentrations of analytes in oral fluid make it impossible to simply apply methods used for urine testing to oral fluid specimen. Measuring the already low analyte concentration in oral fluid samples may be even more challenging due to oral fluid collection procedures during which oral fluid samples are preserved by the addition of certain buffers. Usually this preservation process dilutes the analyte concentration an additional 2 to 4 fold decreasing the analyte concentration further lower (see treated OF, above). Thus, for example, detection of 1-2 ng/ml of THC must be achieved according to the federal guidelines.

Thus, in developing an assay for detecting cannabinoids in oral fluid samples, there are many considerations, not the least of which is sensitivity. Another consideration is interference by materials present in oral fluid samples or interference due to viscosity of the oral fluid sample. Another major obstacle is the fact that Δ⁹-THC and most other cannabinoids are only sparingly soluble in water. The low aqueous solubility of Δ⁹-THC (1-2 ug/ml; Jarho et al., Life Sci. (1998) 63:PL381-4, hereby incorporated by reference) has made it impractical if not impossible to accurately determine the presence or amount of Δ⁹-THC in biological samples, such as oral fluid, where Δ⁹-THC is present only in minute amounts.

Further, currently available oral fluid collection devices pose a common problem of adsorbing most of the hydrophobic analytes, and in particular, hydrophobic cannabinoids, to the collection device and causing an artificially low or negative concentration of the hydrophobic analyte in the oral fluid sample. Thus, for example, Δ⁹-THC, even when present in oral fluid, cannot be accurately be measured. This phenomena is particularly severe for Δ⁹-THC detection in oral fluid due to the unusual hydrophobicity of Δ⁹-THC. For example, collection devices supplied by Immunalysis, Orasure, Cozart, Sardtedt, Randox, and Sciteck are found effectively depleting Δ⁹-THC from oral fluid samples which could result in a false negative assay result.

Cases have been reported that patients who were known to use marijuana constantly, were tested positive for THC when urine samples were analyzed, however, they tested negative when oral fluid samples were analyzed. Studies indicated that almost all of the Δ⁹-THC present in the oral fluid was adsorbed and tightly attached to the collection device during the process of oral fluid collection and thus, became not available for testing by, for example, enzyme immunoassay detection.

Surprisingly, most other analytes, such as opiate, amphetamines, methadone, phencyclidine, cocaine, methadone metabolite, ecstacy, propoxyphene, and others stay in the oral fluid solution and can be retrieved by filtration or centrifugation. Thus, the inability to retrieve the Δ⁹-THC from the collection device seems to be unique to cannabinoids, especially to Δ⁹-THC and due to their hydrophobic nature.

There are numerous organic solvents that can act as a dissolving agent, that are capable of dissolving another substance or that can reverse adsorption of a hydrophobic compound to a hydrophobic surface. Common solvents which dissolve substances or compounds that are insoluble (or nearly insoluble or only partially soluble) in water are acetone, alcohol, formic acid, acetic acid, formamide, BTX (benzene toluene xylene), carbon disulfide, dimethyl sulfoxide, carbon tetrachloride, chloroform, ether, tetrahydrofuran, furfural, hexane and turpentine.

Organic solvents may be classified as polar and non-polar. Polar solvents, like water, have molecules whose electric charges are unequally distributed, leaving one end of each molecule more positive than the other. Usually polar solvents have an O—H bond of which water, methanol, ethanol, and acetic acid are examples. Propanol, butanol, formic acid and formamid are polar solvents. Dipolar solvents which contain a C—O double bond without O—H bond are, for example, acetone ethyl acetate, methyl ethyl ketone, acetonitrile, N,N-dimethylformamide and dimethyl sulfoxide. Non-polar solvents, like carbon tetrachloride, benzene, and diethyl ether, have molecules whose electric charges are equally distributed and are not miscible with water. Exemplary non-polar solvents are hexane, tetrahydrofurn and methylene chloride.

U.S. Pat. No. 6,383,513 and Jarho et al., Life Sci. (1998) 63:PL381-4, both incorporated hereby by reference, disclose the use of cyclodextrins for solubilization of THC.

It is generally known that cyclodextrins form inclusion complexes with various hydrophobic organic or inorganic compounds, and as a result, increase the solubility or stability of these compounds (Duchene et al., Int. J. Pharm. (2003) 266:85-90). The formation of cyclodextrin inclusion complexes with molecules is referred to as the host-guest phenomenon. Cyclodextrins can be regarded as cone-shaped molecules, where the polar hydroxyl groups of the glucose unit are oriented towards the outside of the structure. Therefore, the outside of the cyclodextrins is hydrophilic, whereas the inside of the cavity is hydrophobic giving cyclodextrins the ability to accommodate hydrophobic molecules/moieties in the cavity (Aachmann et al., Protein Eng. (2003) 16:905-912; Bojjinova et al., Carbohydr. Res. (2003) 338:781-785; Kadri et al., Eur. J. Med. Chem. (2004) 39:79-84; Braga et al., Org. Biomol. Chem. 2003) 1:873-878; Bongiorno et al., Carbohydr. Res. (2002) 337:743-754; U.S. Pat. No. 5,134,127; all of which are incorporated herein in their entirety).

This special molecular arrangement accounts for the wide range of beneficial effects cyclodextrins have on different molecules, such as aggregation suppression (if residues responsible for aggregation are highly solvent accessible), protection against degradation (if point of attack is sterically ‘masked’ by cyclodextrin) and alteration of function (if residues involved in function are ‘masked’ by cyclodextrin). The exact effect of cyclodextrins on a given molecule will always be related to the particular structure of the molecule in question (Aachmann et al., Protein Eng. (2003) 16:905-912). A minimum requirement for inclusion complex formation is that the guest molecule must fit at least partially into the cyclodextrin cavity.

Numerous references teach compositions of cyclodextrins with drugs (Okimoto et al., Pharm. Res. (1996) 13:256-264; Loftsson and Brewster, J. Pharm. Sci. (1996) 85:1017-1025; U.S. Pat. No. 5,134,127; U.S. Pat. No. 6,407,079, U.S. Patent Appl. No. 2004/0157796, the disclosure of which are incorporated herein by reference in their entirety). However, as discussed by Aachmann et al. (Protein Eng. (2003) 16:905-912), the interaction or solubilizing capacity of a given cyclodextrin/drug combination is generally unpredictable.

U.S. Pat. No. 6,383,513 discloses the use of cyclodextrin for solubilization of THC in a biphasic delivery system or a microsphere delivery system. The object of the subject matter of U.S. Pat. No. 6,383,513 is the solubilization of THC in order to promote absorption from the nasal cavity. The use of cyclodextrin as a useful method to overcome the poor water solubility of THC was demonstrated by Jarho et al., in their attempt to use THC as a pharmaceutical. Jarho et al. could further improve THC solubility by adding 0.1% hydroxypropylmethylcellulose. However, nothing is disclosed or suggested in U.S. Pat. No. 6,383,513 and in Jarho et al. about retrieving a cannabinoid, such as Δ⁹-THC, from an oral fluid collection device having adsorbed the cannabinoid and wherein the retrieved cannabinoid in the extraction buffer is suitable for direct use in an enzyme immunoassay testing for the presence or absence of the cannabinoid.

A significant advance in the art would be realized if assay systems, methods and kits useful for the qualitatively and quantitatively determination of low concentration of cannabinoids, especially Δ⁹-THC, in oral fluid specimen, could be provided. It is an objective of this invention to overcome the current obstacles and to provide such assay systems, methods and kits to quickly and reliably determine the amount of a cannabinoid in an oral fluid specimen with certain specific, relevant cutoffs. The methods and kits are particularly useful in preserving the Δ⁹-THC quantitatively from oral fluid samples during the process of collection and preparation. The methods and kits provide for retrieving a cannabinoid from an oral fluid collection device employing an extraction buffer comprising DMF or a cyclodextrin or a combination thereof, wherein the cannabinoid retrieved is suitable for direct use in an enzyme immunoassay. In addition, the methods and kits are particularly useful in the detection of recent drug use and for fast determination of cannabinoids using auto-analyzers.

### SUMMARY OF THE INVENTION

It is an objective of this invention to provide methods and kits for the detection of cannabinoids which may be present in minute amounts in an oral fluid.

It is another objective of the present invention to provide methods and kits which are broadly adaptable to a wide variety of automatic analyzers and which will increase the sensitivity in cannabinoid testing by several fold over currently available methods and kits.

While the present invention may be embodied in many different forms, several specific embodiments are discussed herein with the understanding that the present disclosure is to be considered only as an exemplification of the principles of the invention, and it is not intended to limit the invention to the embodiments illustrated.

In its broadest application, the present invention can be used to measure any cannabinoid in any sample. In its narrowest application, the present application provides methods and kits for retrieving Δ⁹-tetrahydrocaninabinol from an oral fluid collection device having adsorbed the Δ⁹-tetrahydrocaninabinol, wherein the retrieved Δ⁹-tetrahydrocaninabinol is suitable for direct use in an enzyme immunoassay.

In a first embodiment of the present invention a method of retrieving a cannabinoid from an oral fluid collection device having adsorbed the cannabinoid is provided, the method comprising the steps of (a) removing the oral fluid from the oral fluid collection device; (b) adding an extraction buffer comprising β-cyclodextrin or DMF to the oral fluid collection device; and (c) removing the extraction buffer from the oral fluid collection device; wherein the retrieved cannabinoid in the removed extraction buffer is suitable for direct use in an enzyme immunoassay testing for the presence or absence of the cannabinoid. The method of the present invention may further comprise the step of (d) using an oral fluid collection device to collect an oral fluid from an individual suspected of having consumed a cannabinoid.

In one embodiment of the present invention, the extraction buffer comprises from about 1% (w/v) to about 2% (w/v) β-cyclodextrin. In another embodiment, the extraction buffer comprises from about 20% (w/v) to about 35% (w/v) DMF. In a preferred embodiment, the extraction buffer comprises a mixture of from about 20% (w/v) to about 35% (w/v) DMF and from about 0.5% (w/v) to about 5% (w/v) β-cyclodextrin.

The β-cyclodextrin may be unmodified or modified. In a preferred embodiment of the present invention, the β-cyclodextrin is a modified β-cyclodextrin selected from the group consisting of hydroxypropyl-β-cyclodextrin and sulfobutyl-ether-β-cyclodextrin. In another embodiment, the extraction buffer comprises a mixture of hydroxypropyl-β-cyclodextrin and sulfobutyl-ether-β-cyclodextrin. The extraction buffer may also comprise a mixture of hydroxypropyl-β-cyclodextrin and β-cyclodextrin or a mixture of sulfobutyl-ether-β-cyclodextrin and β-cyclodextrin. In another embodiment of the present invention, the extraction buffer comprises a mixture of hydroxypropyl-β-cyclodextrin, sulfobutyl-ether-β-cyclodextrin and β-cyclodextrin. The β-cyclodextrin may also be a β-cyclodextrin represented by formula (II).

In one embodiment of the present invention, the extraction buffer is from about 50 mM to about 100 mM Tris and has a pH range of from about 7.2 to about 8.2.

In a preferred embodiment of the present invention, the cannabinoid is Δ⁹-tetrahydrocaninabinol. The cannabinoid may also be a cannabinoid represented by formula (I).

In a preferred embodiment removing the oral fluid from the oral fluid collection device comprises filtration or centrifugation. In another embodiment of the present invention, removing the extraction buffer from the oral fluid collection device comprises compressing the extraction buffer out of the oral fluid collection device.

In a preferred embodiment of the present invention, the enzyme immunoassay is a homogeneous enzyme immunoassay or an ELISA. In a preferred embodiment, the homogeneous enzyme immunoassay comprises glucose-6-phosphate dehydrogenase.

The invention further provides for kits for retrieving a cannabinoid from an oral fluid collection device having adsorbed the cannabinoid, the kit comprising (i) an oral fluid collection device; (ii) an extraction buffer comprising β-cyclodextrin or DMF; and (iii) instructions for retrieving the cannabinoid from the oral fluid collection device.

In one embodiment of the present invention, the kit includes an extraction buffer comprising from about 1% (w/v) to about 2% (w/v) β-cyclodextrin. In another embodiment, the extraction buffer comprises from about 20% (w/v) to about 35% (w/v) DMF. In a preferred embodiment, the kit comprises an extraction buffer comprising a mixture of from about 20% (w/v) to about 35% (w/v) DMF and from about 0.5% (w/v) to about 5% (w/v) β-cyclodextrin.

In a preferred embodiment of the present invention, the kit comprises a β-cyclodextrin that is a modified β-cyclodextrin selected from the group consisting of hydroxypropyl-β-cyclodextrin and sulfobutyl-ether-β-cyclodextrin. In another embodiment, the extraction buffer comprises a mixture of hydroxypropyl-β-cyclodextrin and sulfobutyl-ether-β-cyclodextrin.

In one embodiment of the present invention, the kit comprises an extraction buffer from about 50 mM to about 100 mM Tris and a pH range of from about 7.2 to about 8.2.

In a preferred embodiment of the present invention, the cannabinoid retrieved from the oral fluid collection device is Δ⁹-tetrahydrocaninabinol.

In a preferred embodiment of the present invention, the kit further comprises reagents for performing an enzyme immunoassay to determine the presence or absence of the cannabinoid. The enzyme immunoassay can be a homogeneous enzyme immunoassay or an ELISA. In another preferred embodiment, the homogeneous enzyme immunoassay comprises glucose-6-phosphate dehydrogenase.

### BRIEF DESCRIPTION OF THE DRAWINGS

FIG. 1 depicts a graph showing retrieving a known amount of Δ⁹-THC from a spiked oral fluid sample using an extraction buffer comprising 30% (w/v) DMF. The graph was prepared by plotting the results obtained in Example 4, in which samples containing Δ⁹-THC were assayed according to the present invention. The concentration of Δ⁹-THC (THC) is plotted on the X-axis and absorbance at 340 nm (presented as mA/min) is plotted on the Y-axis. Solid diamonds, reference; solid squares, swab sample; solid triangles, retrieving Δ⁹-THC using extraction buffer comprising 30% (w/v) DMF.

FIG. 2 depicts a graph showing retrieving a known amount of Δ⁹-THC from a spiked oral fluid sample using extraction buffers comprising various concentrations of DMF. The graph was prepared by plotting the results obtained in Example 5, in which samples containing Δ⁹-THC were assayed according to the present invention. The concentration of Δ⁹-THC (THC) is plotted on the X-axis and absorbance at 340 nm (presented as mA/min) is plotted on the Y-axis. Solid diamonds, reference; small solid squares, extraction buffer comprising 25% (w/v) DMF; solid triangles, extraction buffer comprising 30% (w/v) DMF; big solid squares, extraction buffer comprising 35% (w/v) DMF.

FIG. 3 depicts a graph showing retrieving a known amount of Δ⁹-THC from a spiked oral fluid sample using extraction buffers comprising 1% (w/v) hydroxypropyl-β-cyclodextrin (solid squares); 1% (w/v) hydroxypropyl-β-cyclodextrin and 30% (w/v) DMF (solid triangles); 1% (w/v) sulfobutyl-ether-β-cyclodextrin (solid circles); or 1% (w/v) sulfobutyl-ether-β-cyclodextrin and 25% (w/v) DMF (asterisks). The graph was prepared by plotting the results obtained in Example 7, in which samples containing Δ⁹-THC were assayed according to the present invention. The concentration of Δ⁹-THC (THC) is plotted on the X-axis and absorbance at 340 nm (presented as mA/min) is plotted on the Y-axis.

### DEFINITIONS

As used herein:

“About” means “approximately” and refers to a range of values of plus or minus 10% of a specified value. For example, the phrase “about 200” includes plus or minus 10% of 200, or from 180 to 220.

“Adsorb” or “adsorption” refers to the process of one material attracting and holding molecules of another substance to the surface of its molecules. For example, a material, such as an oral fluid collection device, adsorbs another substance, such as a cannabinoid.

“Cannabinoid” refers to a family of compounds that usually contain a pyrane ring, preferably a 1,1′-di-methyl-pyrane ring, a variedly derivatized aromatic ring and a variedly unsaturated cyclohexyl ring and their immediate chemical precursors and derivatives. Particularly included within the term “cannabinoid” are cannabinol derivatives as described in U.S. Patent Appl. No. 20030232101.

“Collection device” refers to a device suitable to collect, harvest or adsorb a cannabinoid that may be present in a biological sample. An oral fluid collection device is a collection device suitable to collect saliva, gingival crevicular fluid and oral mucosal transudate.

“Cyclodextrin” includes α-cyclodextrin, β-cyclodextrin, γ-cyclodextrin as generally depicted in formula (I) as well as molecules where n>6. Included within “cyclodextrin” are derivatives of cyclodextrin, e.g., ether, ester and amide derivatives. Unless otherwise stated, “β-cyclodextrin” refers to unmodified β-cyclodextrin. Particularly included within the term “cyclodextrin” are modified cyclodextrins as described in U.S. Pat. Nos. 5,134,127 and 6,407,079.

“Δ⁸-THC” refers to Δ⁸-tetrahydrocannabinol.

“Δ⁹-THC” refers to Δ⁹-tetrahydrocannabinol.

“Derivative” or “derivatized” or “modified” refers to a compound that is produced from another compound of similar structure by the replacement of substitution of one atom, molecule or group by another. For example, a hydrogen atom of a compound may be substituted by alkyl, acyl, amino, hydroxyl, halo, haloalkyl, etc. to produce a derivative of that compound or a derivatized compound.

“DMF” refers to N,N-dimethylformamide, also known as N-formyldimethylamine.

“Extraction buffer” refers to an aqueous solution capable of retrieving, harvest, collecting or recovering a cannabinoid from a surface having adsorbed the cannabinoid.

Synonyms for the phrase “measuring the amount of a cannabinoid” are contemplated within the scope of the present invention and include, but are not limited to, detecting, measuring, testing, or determining a cannabinoid; detecting, measuring, testing, or determining the presence of a cannabinoid; detecting, measuring, testing, or determining the amount of a cannabinoid; detecting, measuring, testing, or determining the concentration of a cannabinoid.

“Oral fluid” means a biological fluid obtained from the inside of the mouth of an individual. Oral fluid includes saliva, oral mucosal transudate and gingival crevicular fluid.

“w/v” refers to weight/volume.

### INCORPORATION BY REFERENCE

All publications and patent applications cited in this specification are herein incorporated by reference as if each individual publication or patent application were specifically and individually indicated to be incorporated by reference.

### DETAILED DESCRIPTION OF THE INVENTION

The present invention provides methods and kits for retrieving a cannabinoid from an oral fluid collection device having adsorbed the cannabinoid is provided, the method comprising the steps of (a) removing the oral fluid from the oral fluid collection device; (b) adding an extraction buffer comprising β-cyclodextrin or DMF to the oral fluid collection device; and (c) removing the extraction buffer from the oral fluid collection device; wherein the retrieved cannabinoid in the removed extraction buffer is suitable for direct use in an enzyme immunoassay testing for the presence or absence of the cannabinoid. The method of the present invention may further comprise step (d) collecting an oral fluid from an individual suspected of having consumed a cannabinoid using an oral fluid collection device.

The following will describe the individual components and parameters of the homogenous enzyme immunoassay in detail.

I. Removing the Oral fluid from the Oral Fluid Collection Device

### A. Oral fluid Collection Device

The present invention provides methods and kits for retrieving a cannabinoid from an oral fluid collection device having adsorbed the cannabinoid, the method comprising the step of removing the oral fluid from the oral fluid collection device.

The oral fluid that is collected from the inside of the mouth of an individual comprises oral mucosal transudate, gingival crevicular fluid or saliva. Oral mucosal transudate is an ultra-filtrate of blood which is ideal for drug testing, especially cannabinoids. Oral mucosal transudate comes from the cheek and gums. Saliva is a clear, tasteless, odorless, slightly alkaline fluid secreted into the mouth by the salivary glands.

Oral fluid can be collected quickly and painlessly from the inside of the mouth of an individual eliminating the inconvenience associated with collecting blood or urine samples.

As an alternative of spitting an oral fluid into a container, typically oral fluid collection devices are used to collect oral fluid from an individual. These devices are designed to allow an individual placing the device into the mouth to adsorb an oral fluid sample for convenience and ease of use. The oral fluid collection device may be attached to a nylon stick or the like, which allows easy placement in the gingival crevice.

However, hydrophobic organic compounds, such as Δ⁹-THC and other cannabinoids, that may be present in the oral fluid, readily stick to surfaces of collection devices such as from normal glass, plastic, cotton, cellulose membranes, most of organic polymers, and the like (See Example 1).

Examples of commercially available oral fluid collection devices include those supplied by Immunalysis, Orasure, Cozart, Sardtedt, Randox, and Sciteck. Other oral fluid collection devices include the Orasure® and Intercept® collection systems (OraSure Technologies, Inc.; Altrix), consisting of a cotton fiber pad.

Oral fluid collection devices can consist of cellulosic paper or woven cellulosic filaments in the form of a fabric with weft and warp threads or can be in the form of an unwoven fabric. It is preferred to use natural cellulosic material or synthetic resin fabric of monofile or spun filaments which can consist of cellulose materials, for example cotton, cellulose, flax or sisal. The collection device may be a cotton swab or cotton pad.

Oral fluid collection devices usually are solid and have no or very little solubility in water or in the extraction buffer of the present invention. The oral fluid collection device can be shaped in particles, plates, fiber, hollow fiber and the like, and the shape and size are not particularly limited. Oral fluid collection devices may be of any shape and size that fit into the mouth of an individual from whom an oral fluid sample is to be collected. Oral fluid collection devices also include devices that, usually, are not placed into the mouth of an individual, such as filters, beakers and other containers suitable to collect an oral fluid sample from an individual. The oral fluid collection device may have numerous pores of suitable size. The more porous the structure, the better the adsorption ability per unit volume of oral fluid.

The oral fluid collector may be impregnated with a hypertonic saline solution.

Oral fluid can be collected anytime and almost anywhere either by, for example, a trained member of staff or an external collector. To collect a cannabinoid from an oral fluid of a person suspected of having consumed a cannabinoid, generally, an oral fluid collection device, for example, a cotton swab or pad, is placed into an individual's mouth for sufficient time to allow adsorption of the individual's oral fluid to the oral fluid collection device. The oral fluid collection device may be wedged between the lower cheek and gum or swabbed around the upper and lower outer gums. Alternatively, the individual provides an oral fluid sample by spitting the oral fluid into or onto an oral fluid collection device.

The time for placing an oral fluid collection device into an individuals' mouth can vary. Typically, the time is for at least about 10 seconds, preferably at least about 30 seconds, more preferably at least about 1 minute, still more preferably at least about 2 minute, yet more preferably at least about 5 minutes. The oral fluid collection device may be placed in an individual's mouth for up to 15 minutes.

During this period of time that individual's oral fluid is brought into contact with the oral fluid collection device and the cannabinoid present in the oral fluid can be adsorbed by the oral fluid collection device.

The oral fluid sample may be collected from an individual and analyzed shortly thereafter using the methods of the present invention. Alternatively, the oral fluid sample may be stored at appropriate temperatures until further analysis. The oral fluid sample may also be frozen for long-term storage. Upon long-term storage, usually a preservatives as known in the art is added to the oral fluid sample. A stabilizer may be added to the oral fluid sample. Such stabilizers may include, but are not limited to, proteins, such as albumin, and surfactants, such as non-ionic surfactants, binding enhancers, e.g., polyalkylene glycols, or the like.

In addition to oral fluid, the methods and kits of the present invention are also applicable to other biological fluid samples, including whole blood, serum, plasma, urine, tears, mucus ascites fluid, oral fluid, lymph, semen, stool, sputum, arthral fluid, cerebrospinal fluid and fetal fluid.

### B. Cannabinoid

The present invention provides methods and kits for retrieving a cannabinoid from an oral fluid collection device having adsorbed the cannabinoid.

Cannabinoids useful in the present invention include, but are not limited to, natural cannabinoids, Δ⁹-THC, Δ⁸-THC, cannabidiol, olivetol, cannabinol, cannabigerol, nabilone, cannabichromene, cannabidivarol, tetrahydrocannabidiol, tetrahydrocannabigerol, tetrahydrocannabichromene, tetrahydrocannabidivarol, the carboxylic acid precursors of the foregoing compounds, and related naturally occurring compounds and their derivatives. “Natural cannabinoid” includes non-natural derivatives of cannabinoids which can be obtained by derivatization of natural cannabinoids.

In a preferred embodiment of the present invention, the cannabinoid is a cannabinol, preferably, Δ⁹-tetrahydrocananinabiol (THC).

In another preferred embodiment of the present invention, the cannabinoid is a cannabinol, preferably, Δ⁸-tetrahydrocananinabiol (THC).

Included within the term of cannabinoid as used herein, are cannabinols or derivatives thereof as described in U.S. Patent Appl. No. 20030232101 (the disclosure of which is incorporated herein in its entirety) and has a structure represented by formula (I):

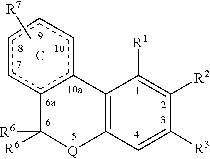

wherein,

R¹ is:

a) H,

b) a C₁₋₄ alkyl group or ester thereof,

c) COOH,

d) OH,

e) a O—C₁₋₅ alkyl (preferably OCH₃) or alkanoyl, optionally substituted by mono- or di-methylamino or ethylamino groups,

f) a O—CO—C₃₋₁₀ alkyl group containing a carboxyl or amino group, O—CO—(H₂C)ₙ— 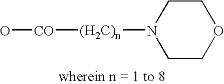
wherein n=1 to 8

g)

h) a p-aminobenzyl group or a C₁₋₇ aminoalkyl group or an organic or mineral acid addition salt thereof, an isocyanate or isothiocyanate derivative of the p-aminobenzyl or aminoalkyl group, a carboxyl terminated derivative of the aminoalkyl group having from 1 to 7 additional carbon atoms or a salt thereof, and an activated derivative of the carboxyl terminated derivative;

i) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen;

j) a lactone (e.g., COCOH); or

k) CH(CH₃)CO₂H or —OCOCH₃

R² is:

a) H, OH, COOH, or a halogen

b) C₁₋₆ carboxy or alkoxy group, or

c) R¹ and R² comprise a substituent of the formula —O(CH₂)₃₋₅, wherein R¹ and R², together with the carbon atoms to which they are bonded, comprises a ring where at least one hydrogen atom thereof is optionally substituted with a halogen.

R³ is:

a) (W)ₘ—Y-(Z)ₙ, wherein

W is a C₅₋₁₂ straight or branched (preferably 1S′CH₃, 2R′CH₃ dimethyl) alkyl, alkenyl, alkynyl, group, or mixture thereof, optionally substituted with at least one halogen,

Y is a bond, O, S, SO, SO₂, CO, NH, N(C₁₋₆ alkyl), or NCS,

Z is:

i) a C₅₋₁₂ alkyl, alkenyl, alkynyl, group, or mixture thereof, optionally substituted with at least one halogen, optionally substituted with a terminal aromatic ring,

ii) CN₁₋₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

iii) a phenyl or benzyl group, optionally substituted with halo, C₁₋₆ alkyl, C₁₋₆ alkoxy, C₁₋₆ alkylthio, CN, CF₃, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, and wherein

m and n are the same or different, and each is either 0 or 1,

b) a C₅₋₁₂ alkyl or haloalkyl group, optionally substituted with a terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different, or

c) a C₅₋₁₂ alkene or alkyne group, optionally substituted with a halogen, dithiolene, terminal aromatic ring, CN₁₋₃, NCS, CO₂H, or CO₂C₁₋₄ alkyl, CONH₂, CONHC₁₋₄ alkyl, or CON(C₁₋₄ alkyl)₂, wherein each C₁₋₄ alkyl on the amide nitrogen can be the same or different;

R⁶ and R^{6′} together form ═O or ═S, or each is independently selected from the group consisting of:

a) hydrogen,

b) C₁₋₆ alkoxy, C₁₋₆ alkylthio, C₁₋₆ alkyl, or C₁₋₆ haloalkyl,

c) CN,

d) CO₂H,

e) CO₂—C₁₋₄ alkyl,

f) C(Y)(Z)-OH,

g) C(Y)(Z)-O—C₁₋₄ alkyl, and

h) C₁₋₆ alkyl-CO₂—Y,

wherein Y and Z are each independently H or C₁₋₆ alkyl,

R⁷ is:

a) hydroxy or lactone,

b) halo,

c) C₁₋₆ alkoxy, C₁₋₆ alkylthio, C₁₋₆ alkyl, or C₁₋₆ haloalkyl,

d) CN,

e) N₃,

f) CO₂H,

g) CO₂—C₁₋₄ alkyl,

h) C(Y)(Z)-OH,

i) C(Y)(Z)-O—C₁₋₄ alkyl,

j) C₁₋₆ alkyl-CO₂—Y, or

k) ═O or ═S,

wherein Y and Z are each independently H or C₁₋₆ alkyl;

Q is:

a) O or S, or

b) N—W, wherein

W is:

i) hydrogen,

ii) C₁₋₆ alkoxyalkyl, C₁₋₆ alkyl, or C₁₋₆ haloalkyl

iii) OC₁₋₆ alkyl, or OC₁₋₆ haloalkyl,

iv) CN,

v) C₁₋₆ alkyl,

vi) C(Y)(Z)C₁₋₄ alkyl, or

vii) C₁₋₆ alkyl-CO₂-Z,

wherein Y and Z are each independently H or C₁₋₆ alkyl.

While R⁷ can be at any of positions 7-10 of ring C, preferably it is at position 9 of the ring.

Ring C in Formula II can be any of the following (the dashed lines representing a double bond at either the Δ6a-10a, Δ8-9, or Δ9-10 position):

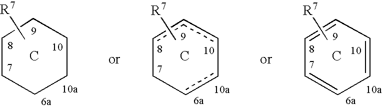

Many compounds according to Formula II are well known, and others can be manufactured in accordance with published methods (see, for example, International Patent Appl. WO99/20268 (Burstein), and U.S. Pat. No. 2,509,386 (Adams), U.S. Pat. No. 3,799,946 (Loev), U.S. Pat. No. 3,856,821 (Loev), U.S. Pat. No. 3,897,306 (Vidic et al.), U.S. Pat. No. 4,064,009 (Fukada et al.), U.S. Pat. No. 4,087,545 (Archer et al.), U.S. Pat. No. 4,142,139 (Bindra), U.S. Pat. No. 4,309,545 (Johnson), U.S. Pat. No. 4,599,327 (Ngrdi et al.), U.S. Pat. No. 4,833,073 (McNally et al.), U.S. Pat. No. 4,876,276 (Mechoulan et al.), U.S. Pat. No. 4,973,603 (Burstein), U.S. Pat. No. 5,338,753 (Burstein et al.), U.S. Pat. No. 5,389,375 (ElSohly), U.S. Pat. No. 5,440,052 (Makriyannis et al.), U.S. Pat. No. 5,605,906 (Lau), and U.S. Pat. No. 5,635,530 (Mechoulam et al.); and Charalambous et al., Pharm. Biochem. Behav., 40, 509-12 (191), Gareau et al., Bioorg. Med. Chem. Lett., 6(2), 189-94 (1996), Griffin et al., Br. J. Pharmacol., 126, 1575-84 (1999), Huffman et al., Bioorg. Med. Chem. Lett., 6, 2281-88 (1998), Lemberger et al., Clin. Pharmacol. Ther., 18(6), 720-26 (1975), Loev et al., J. Med. Chem., 16(11), 1200-06 9 (1973), Loev et al., J. Med. Chem., 17(11), 1234-35 (1974), Martin, et al., Pharm. Biochem. Behav., 46, 295-301 (1993), Papahatjis et al., J. Med. Chem., 41(7), 1195-1200 (1998), Pars et al., J. Med Chem., 19(4), 445-53 (1976), Pertwee et al., Pharmacol. Ther., 74(2),129-80 (1997), Razdan et al., J. Med. Chem., 19(4), 454-60 (1976), Razdan, Pharmacol. Reviews, 38(2) 75-149 (1980), Reggio et al., J. Med. Chem., 40(20), 3312-18 (1997), Reggio et al., Life Sci., 56(23/24), 2025-32 (1995), (Ross et al., Br. J. Pharmacol., 126, 665-72 (1999), Thomas et al., J. Pharm. Exp. Ther., 285(1), 285-92 (1998), Wiley et al., J. Pharm. Exp. Ther., 285(1), 995-1004 (1998), Winn et al., J. Med. Chem., 19(4), 461-71 (1976), and Xie et al., J. Med Chem., 41, 1-67-74 (1998)). ,

### C. Removing the Oral Fluid

The present invention provides methods and kits for retrieving a cannabinoid from an oral fluid collection device having adsorbed the cannabinoid, the method comprising the step of removing the oral fluid from the oral fluid collection device. Specifically, once the individual's oral fluid is adsorbed to the oral fluid collection device, as discussed above, the oral fluid collection device is taken from that individual's mouth and the oral fluid collected is removed from the oral fluid collection device.

During this ‘removal’ process, most drugs, other than Δ⁹-THC and other cannabinoids, can be retrieved efficiently from the oral fluid collection device by filtration or centrifuging. However, Δ⁹-THC and other hydrophobic cannabinoids remain adsorbed to the oral fluid collection device and are almost completely removed from the oral fluid by filtering or centrifuging (See Example 1).

In one embodiment of the invention, removing the oral fluid from the oral fluid collection device is by filtration.

In another embodiment of the invention, removing the oral fluid from the oral fluid collection device is by centrifugation.

In one embodiment of the invention, removing the oral fluid from the oral fluid collection device is by compressing the oral fluid out of the oral fluid collection device. This can be done manually by squeezing, condensing, squashing the oral fluid collection device and/or by applying pressure to it.

Removing the oral fluid from the oral fluid collection device, usually leaves the oral fluid collection device semi-dry.

II. Adding Extraction Buffer Comprising P-Cyclodextrin or N,N-Dimethylformamide to the Oral Fluid Collection Device

### A. Extraction Buffer

The present invention provides methods and kits for retrieving a cannabinoid from an oral fluid collection device having adsorbed the cannabinoid, the method comprising the step of adding an extraction buffer comprising β-cyclodextrin or N,N-dimethylformide to the oral fluid collection device. Specifically, once the oral fluid is removed from the oral fluid collection device, as described above, an extraction buffer is added to the oral fluid collection device to retrieve the cannabinoid from the collection device having adsorbed the cannabinoid. The retrieved cannabinoid should be suitable for direct use in an enzyme immunoassay. Thus, the pH and components of the extraction buffer need to be evaluated to ensure accurate and reliable performance of the enzyme immunoassays.

Typically, the extraction buffer is added to a semi-dry oral fluid collection device (see herein) and is incubated with the oral fluid collection device for a sufficient time to allow retrieving the cannabinoid from the oral fluid collection device. The longer the extraction buffer is incubated with the oral fluid collection device having adsorbed a cannabinoid, the more quantitatively the cannabinoid can be retrieved.

Various buffers may be used to achieve the desired pH and maintain the desired pH during most enzyme immunoassays. Illustrative buffers include borate, phosphate, carbonate, tris, barbital and the like. However, not all buffers are suitable analyzing the cannabinoid concentration in oral fluid samples. Particularly, some of these buffer ingredients are not desirable for homogenous enzyme immunoassays employing G6PDH. Thus, it is an objective of this invention to provide an extraction buffer suitable for retrieving a cannabinoid from an oral fluid collection device having adsorbed the cannabinoid and for testing for the presence or absence of the cannabinoid by directly using an enzyme immunoassay, preferably a homogeneous enzyme immunoassay employing G6PDH.

In a preferred embodiment of this invention, the extraction buffer contains tris (Tris-(hydroxymethyl)-aminomethane. Usually, the final concentration of tris in the homogeneous enzyme immunoassays is in the range of 50-200 mM, preferably in the range of 75-150 mM, more preferably in the range of 80-100 mM.

Using the extraction buffer of this invention the pH in the homogeneous enzyme immunoassays can be adjusted to a final pH range from between 7.2 to 8.3, which is suitable for homogeneous enzyme immunoassays using G6PDH.

In a preferred embodiment of the invention, the extraction buffer has a buffer capacity of from about 50 mM to about 100 mM and has a pH in the range of from about 7.0 to about 9.0. In another embodiment of this invention, the pH of the extraction buffer is in the range of from about 7.5 to about 8.5. In one embodiment of the invention, the pH of the extraction buffer is in the range of from about 7.2 to about 8.2.

The time for incubating the extraction buffer with the oral fluid collection device can vary. Typically, the time for incubation is for at least about 30 seconds, preferably at least about 60 seconds, more preferably at least about 5 minutes, still more preferably at least about 10 minute, yet more preferably at least about 15 minutes, even more preferably at least 30 minutes. Illustratively, the time, the oral fluid collection device is placed into an individual's mouth from about 30 seconds to about 30 minutes, preferably from about 30 seconds to about 15 minutes, more preferably from about 1 minute to about 15 minutes, still more preferably about 1 minute to about 10 minutes, yet more preferably from about 2 minute to about 10 minute and even more preferably from about 2 minutes to about 5 minutes. It is understood that the incubation time may vary and may depend on the, for example, hydrophobic nature of the cannabinoid in question that is to be retrieved from the oral fluid collection device.

Moderate temperatures are normally employed for retrieving a cannabinoid from an oral fluid collection device having adsorbed the cannabinoid. Acceptable temperatures employed in the methods of this invention will be in the range of from about 4° C. to about 40° C., more usually in the range of from about 10° C. to about 40° C., preferably in the range of from about 20° C. to about 40° C., most preferably in the range of from about 20° C. to about 25° C. In a preferred embodiment of the present invention, the temperature is room temperature.

### B. Extraction Buffer Comprising an Organic Solvent

Hydrophobic organic compounds are known to attach to hydrophobic solid surfaces, dissolve in organic solvents, immiscible with hydrophilic liquids. A large number of drugs, including cannabinoids are only poorly or sparingly soluble in water. If the drug molecule has basic or acidic groups there exists the further possibility of increasing the water solubility by salt formation. However, this usually results in decreased efficacy or impaired chemical stability.

It is an objective of the present invention to provide methods and kits for retrieving a cannabinoid, especially, Δ⁹-THC, from an oral fluid collection device having adsorbed the cannabinoid, the method comprising an extraction buffer comprising an organic solvent, wherein the retrieved cannabinoid in the removed extraction buffer is suitable for direct use in an enzyme immunoassay testing for the presence or absence of the cannabinoid. Because enzymatic activity, such as glucose-6-phosphatase dehydrogenase activity, can be drastically impacted by components of an extraction buffer, it is not obvious and rather unpredictable which organic solvents will work in the enzyme immunoassays. Thus, whatever solvent is employed for retrieving Δ⁹-THC or other cannabinoid from the oral fluid collection device, it should be compatible with the enzyme immunoassay.

As such, the organic solvent used for retrieving Δ⁹-THC or other cannabinoid should not interfere with the interaction among the enzyme, antibody and cannabinoid (as described herein and in U.S. patent application Ser. No. 10/927,823). Further, because the enzyme immunoassay is carried out using an assay buffer system (as detailed herein), the organic solvent must be compatible and miscible with this assay buffer system. Thus, a solvent suitable for the methods and kits of the present invention, must meet the following conditions: (1) retrieve Δ⁹-THC or other cannabinoid from an oral fluid collection device; (2) not interfere with the enzyme-antibody-cannabinoid interaction; and (3) be compatible, i.e., miscible, with the assay buffer system. Quite surprisingly, it has been found that certain organic solvents meet the above conditions and can be used to retrieve cannabinoids, especially Δ⁹-THC, form an oral fluid collection device and allow the direct use of an enzyme immunoassay to test for the presence or absence of the retrieved cannabinoid in the extraction buffer (See Examples 2, 3).

Organic solvents which are suitable for use in the methods of present invention are solvents which are mixable with water such as dimethylsulfoxide (DMSO), N,N-dimethylformamide (DMF), dioxane, acetonitrile, ethylacetate and lower alcohols. Lower alcohols are C₁-C₆ alcohols, wherein the alkyl chain may be branched or unbranched. Other suitable alcohols are C₂-C₄ alcohols such as ethanol, and n-propyl alcohol.

In one embodiment, the extraction buffer comprises DMSO. A suitable amount of DMSO in the extraction buffer is about 30% (w/v) which is suitable to retrieve <10% of Δ⁹-THC from a cotton swab (See Example 3).

In one embodiment, the extraction buffer comprises ethanol. A suitable amount of ethanol in the extraction buffer is about 30% (w/v) which is suitable to retrieve <5% of Δ⁹-THC from a cotton swab (See Example 3).

In one embodiment, the extraction buffer comprises dioxane. A suitable amount of dioxane in the extraction buffer is about 30% (w/v) which is suitable to retrieve <10% of Δ⁹-THC from a cotton swab (See Example 3).

### C. Extraction Buffer Comprising N,N-Dimethylformamide (DMF)

In a preferred embodiment of the present invention, the extraction buffer used to retrieve a cannabinoid from an oral fluid collection device having adsorbed the cannabinoid comprises N,N-dimethylformamide (DMF).

DMF is a colorless, high-boiling, mobile, polar liquid with a faint, characteristic odor. It is freely miscible with water, alcohols, ethers, ketones, esters, carbon disulfide and chlorinated and aromatic hydrocarbons. It is either immiscible or only partly miscible with aliphatic hydrocarbons. DMF is considered an aprotic solvent with a high dielectric constant. The high solvent power of DMF can be ascribed to its molecular structure. DMF is a very suitable solvent for salts or compounds with a high molecular weight owing to the combined action of its small molecule, its high dielectric constant, its electron donor properties, and its ability to form complexes (BASF, Technical Data Sheet for DMF).

DMF is present in a total amount of about 15% (w/v) to about 50% (w/v), preferably in a total amount of about 15% (w/v) to about 45% (w/v), more preferably in a total amount of about 15% (w/v) to about 40% (w/v), still more preferably about 25% (w/v) to about 35%.

The efficiency of retrieving an adsorbed cannabinoid from an oral fluid collection device with DMF is depending on the concentration of DMF. In one embodiment, the extraction buffer comprises 15% (w/v) DMF, which is suitable to retrieve <10% of Δ⁹-THC from a cotton swab (See Example 3). In other embodiments of the present invention, the extraction buffer comprises 25% (w/v), 30% (w/v), or 35% (w/v) DMF, which is suitable to retrieve >95% of Δ⁹-THC from a cotton swab (See Examples 3-5).

### D. Extraction Buffer Comprising β-Cyclodextrin

In one embodiment of the present invention, the extraction buffer used to retrieve a cannabinoid from an oral fluid collection device having adsorbed the cannabinoid, comprises a cyclodextrin, preferably a β-cyclodextrin.

Cyclodextrins are a group of homologue cyclic oligosaccharides consisting of six, seven or eight α-D-glucopyranose units linked at the 1,4 positions by α-linkages. Cyclodextrins are respectively called α-, β-, or γ-cyclodextrin. Cyclodextrins are generally represented by the schematic formula (II),

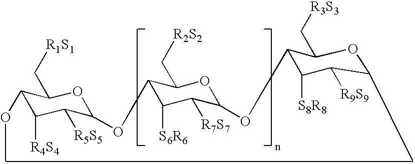

wherein RS represent hydroxyl groups in the 2,3, and 6-positions of the glucopyranose units. When n=4, the molecule is commonly known as α-cyclodextrin or cyclohexaamylose. When n=5, the molecule is commonly known as β-cyclodextrin or cycloheptaamylose. When n=6, the molecule is commonly known as γ-cyclodextrin or cyclooctaamylose.

The methods and kits of the present invention comprise at least one cyclodextrin, also referred to herein as a cyclodextrin derivative or modified cyclodextrin. Examples of the cyclodextrin which can be employed in the methods and kits of the present invention include α-cyclodextrin, β-cyclodextrin and γ-cyclodextrin. Preferably, the cyclodextrin is a cyclodextrin.

β-cyclodextrin forms efficiently complexes with Δ⁹-THC. Seemingly, Δ⁹-THC fits perfectly into the cavity formed by β-cyclodextrin. The cavities formed by α-cyclodextrin and γ-cyclodextrin are smaller or larger, respectively, when compared to the cavity formed by β-cyclodextrin. Thus, the cavities of α-cyclodextrin and γ-cyclodextrin may not be perfectly sized to accomodate Δ⁹-THC. However, α-cyclodextrin and γ-cyclodextrin may be more suitable than β-cyclodextrin to retrieve from an oral fluid collection device cannabinoids other than Δ⁹-THC that may fit better into the cavities of α-cyclodextrin or γ-cyclodextrin.

The β-cyclodextrin may be unmodified or modified. In one embodiment of the present invention, the cyclodextrin is a partially etherified β-cyclodextrin, substantially as is described in U.S. Pat. No. 6,407,079 to Muller et al., of formula (III):
(β-cyclodextrin)-(OR)₂₁ (III)

wherein R groups are independently selected from hydrogen, hydroxyalkyl or alkyl and wherein at least one R group is hydroxyalkyl. Preferably, the at least one hydroxyalkyl group is hydroxyethyl, hydroxypropyl or dihydroxypropyl. Preferred alkyl groups are methyl and/or ethyl groups.

β-cyclodextrin is a compound with ring structure consisting of 7 anhydro glucose units; it is also referred to as cycloheptaamylose. Each of the 7 glucose rings contains in 2-, 3-, and 6-position three hydroxy groups which may be etherified. Therefore, a total of 21 hydroxy groups per cyclodextrin molecule are available for etherification. In the partially etherified β-cyclodextrin derivatives suitable for the methods and kits of the present invention only a portion of these available hydroxy groups are etherified with hydroxyalkyl groups. Optionally, a portion of these available hydroxy groups are etherified with alkyl groups.

Especially preferred β-cyclodextrins are hydroxyethyl, hydroxypropyl and dihydroxypropyl ether β-cyclodextrins, their corresponding mixed ethers, and further mixed ethers with methyl or ethyl groups, such as methyl-hydroxyethyl, methyl-hydroxypropyl, ethyl-hydroxyethyl, and ethyl-hydroxypropyl ether of β-cyclodextrin.

In a preferred embodiment of the present invention, the modified β-cyclodextrin is hydroxypropyl-β-cyclodextrin. At least two commercial preparations of hydroxypropyl-β-cyclodextrin are available, Encapsin® and Molecusol®.

Preparation of hydroxyalkyl ethers of β-cyclodextrin can be carried out using any suitable method, for example methods described in U.S. Pat. No. 3,459,731.

In another embodiment of the present invention, the β-cyclodextrin is a partially alkylated β-cyclodextrin, for example a partially methylated or partially dimethylated β-cyclodextrin.

In another embodiment, the cyclodextrin is selected from those described in U.S. Pat. No. 5,134,127, the disclosure of which is hereby incorporated in its entirety, and has a structure represented by formula (II), wherein:

n is 4, 5, or 6;

R₁, R₂, R₃, R₄, R₅, R₆, R₇, R₈ and R₉ are each, independently, O⁻ or a O—(C₂₋₆-alkylene)-SO₃⁻ group, wherein at least one of R₁ and R₂ is independently a O—(C₂₋₆-alkylene)-SO₃⁻ group, preferably a O—(CH₂)ₘ SO₃⁻ group, wherein m is 2 to 6, preferably 2 to 6, preferably 2 to 4, (e.g. OCH₂CH₂CH₂SO₃⁻ or OCH₂CH₂CH₂CH₂SO₃⁻); and S₁, S₂, S₃, S₄, S₅, S₆, S₇, S₈, and S9 are each, independently, a pharmaceutically acceptable cation which includes, for example, H⁺, alkali metals (e.g. Li⁺, Na⁺, K⁺), alkaline earth metals (e.g., Ca⁺², Mg⁺²), ammonium ions and amine cations such as the C₁₋₆ alkylamines, piperidine, pyrazine, C₁₋₆ alkanolamine and C₄₋₈ cycloalkanolamine.

In a preferred embodiment of the present invention, R₁ is a O—(C₂₋₆-alkylene)-SO₃⁻ group, more preferably a O—(CH₂)ₘ SO₃⁻ group wherein m is 2-6, preferably 2-4 (e.g. OCH₂CH₂CH₂SO₃⁻ or OCH₂CH₂CH₂CH₂SO₃⁻); R₂ to R₉ are O⁻; and S₁ to S₉ are each, independently, a pharmaceutically acceptable cation.

In another preferred embodiment of the present invention, R₁, R₂ and R₃ are each, independently, a O—(C₂₋₆-alkylene)-SO₃⁻ group, preferably a O—(CH₂)ₘ SO₃⁻ group wherein m is 2-6, preferably 2-4 (e.g. OCH₂CH₂CH₂SO₃⁻ or OCH₂CH₂CH₂CH₂SO₃⁻); R₄ to R₉ are O⁻; and S₁ to S₉ are each, independently, a pharmaceutically acceptable cation.

In yet another preferred embodiment of the present invention, R₁, R₂ and R₃ are each, independently, a O—(C₂₋₆-alkylene)-SO₃⁻ group; at least one of R₄, R₆, and R₈ is a O—(C₂₋₆-alkylene)-SO₃⁻ group, preferably a O—(CH₂)ₘ SO₃⁻ group wherein m is 2-6, preferably 2-4 (e.g. OCH₂CH₂CH₂SO₃⁻ or OCH₂CH₂CH₂CH₂SO₃⁻); R₅, R₇ and R₉ are O⁻; and S₁ to S₉ are each, independently, a pharmaceutically acceptable cation.

In another preferred embodiment of the present invention, R₁, R₂, R₃, R₄, R₆ and R₈ are each, independently, a O—(C₂₋₆-alkylene)-SO₃⁻ group; at least one of R₄, R₆, and R₈ is a O—(C₂₋₆-alkylene)-SO₃⁻ group, preferably a O—(CH₂)ₘ SO₃⁻ group wherein m is 2-6, preferably 2-4 (e.g. OCH₂CH₂CH₂SO₃⁻ or OCH₂CH₂CH₂CH₂SO₃⁻); R₅ R₇ and R₉ are O⁻; and S₁ to S₉ are each, independently, a pharmaceutically acceptable cation.

Preferred among these cyclodextrin derivatives are those wherein the C₂₋₆ alkylene is a C₃ or C₄ alkylene. A particularly preferred cyclodextrin is sulfoalkylether-β-cyclodextrin, for example sulfobutyl-ether-β-cyclodextrin. In a preferred embodiment of the present invention, the modified β-cyclodextrin is sulfobutyl-ether-β-cyclodextrin. Sulfobutyl-ether-β-cyclodextrins exhibit good water solubilities and effective complexation characteristics at all levels of substitution. A hepta-substituted preparation (Captisol, marketed by Cydex) has high intrinsic aqueous solubility (>50% w/v).

In another embodiment of the present invention, the cyclodextrin is an ester derivative (acylates, sulfonates, sulfates and phosphates).

For ease of formulation, inclusion efficiency, economical reasons and commercial availability, the cyclodextrin is preferably a β-cyclodextrin, more preferably a β-cyclodextrin alkylated or hydroxyalkylated in the 2-, 3- and/or 6-position. Particularly useful β-cyclodextrins include hydroxylpropyl-β-cyclodextrin and heptakis-(2,6-di-O-methyl)-β-cyclodextrin.

In one embodiment of the present invention, the extraction buffer comprises at least one cyclodextrin to retrieve a cannabinoid from an oral fluid collection device having adsorbed the cannabinoid. In another embodiment of the present invention, the extraction buffer comprises more than one cyclodextrin. In a preferred embodiment of the present invention, the extraction buffer comprises a mixture of hydroxylpropyl-β-cyclodextrin and sulfobutyl-ether-β-cyclodextrin. In another preferred embodiment of the present invention, the extraction buffer comprises a mixture of hydroxypropyl-β-cyclodextrin and β-cyclodextrin or a mixture of sulfobutyl-ether-β-cyclodextrin and β-cyclodextrin. In another embodiment of the present invention, the extraction buffer comprises a mixture of hydroxypropyl-β-cyclodextrin, sulfobutyl-ether-β-cyclodextrin and β-cyclodextrin.

The amount of cyclodextrin to be employed in the methods and kits of the present invention is generally in an amount of at least about 0.5% (w/v), preferably at least about 1% (w/v), more preferably at least about 2% (w/v), still more preferably at least about 5% (w/v), yet more preferably at least about 10% (w/v), and even more preferably at least about 15% (w/v). Illustratively, a cyclodextrin is present in a total amount of about 0.5% (w/v) to about 50% (w/v), preferably in a total amount of about 0.5% (w/v) to about 20% (w/v), more preferably in a total amount of about 1% (w/v) to about 15% (w/v), still more preferably about 1% (w/v) to about 10% (w/v), yet more preferably in a total amount of about 2% (w/v) to about 10% (w/v) and even more preferably in a total amount of about 2% (w/v) to about 5% (w/v).

Modified β-cyclodextrins are more soluble than unmodified β-cyclodextrins. Thus, modified β-cyclodextrins may be used at higher concentrations (w/v) than unmodified β-cyclodextrin. Illustratively, a modified β-cyclodextrin is present in a total amount of about 0.5% (w/v) to about 80% (w/v), preferably in a total amount of about 0.5% (w/v) to about 60% (w/v), more preferably in a total amount of about 1% (w/v) to about 50% (w/v), still more preferably about 1% (w/v) to about 40% (w/v), yet more preferably in a total amount of about 2% (w/v) to about 30% (w/v) and even more preferably in a total amount of about 2% (w/v) to about 2% (w/v).

### E. Extraction Buffer Comprising β-Cyclodextrin and DMF

In another embodiment of the present invention, the extraction buffer comprises DMF and a cyclodextrin, preferably a β-cyclodextrin. Quite surprisingly, it was found that unmodified β-cyclodextrin in combination with DMF works very well in retrieving a cannabinoid from an oral fluid collection (see Example 6). Thus, in a preferred embodiment of the present invention, the extraction buffer comprises a mixture of from about 20% (w/v) to about 35% (w/v) DMF and from about 0.5% (w/v) to about 5% (w/v) β-cyclodextrin. Other concentrations of β-cyclodextrin and of DMF, as disclosed herein, can be used in combination with each other. The β-cyclodextrin may be unmodified or modified, for example, hydroxypropyl-β-cyclodextrin (See Example 6) or sulfobutyl-ether-β-cyclodextrin (See Example 6).

III. Removing the Extraction Buffer from the Oral Fluid Collection Device

The present invention provides methods and kits for retrieving a cannabinoid from an oral fluid collection device having adsorbed the cannabinoid, the method comprising the step of removing the extraction buffer from the oral fluid collection device. Thus, after incubating the extraction buffer with the oral fluid collection device, as described above, the extraction buffer is removed.

In a preferred embodiment of the invention, removing the extraction buffer from the oral fluid collection device is by compressing the oral fluid out of the oral fluid collection device. This can be done manually by squeezing, condensing, squashing the oral fluid collection device and/or by applying pressure to it.

In one embodiment of the invention, removing the extraction buffer from the oral fluid collection device is by filtration.

In another embodiment of the invention, removing the extraction buffer is removed from the oral fluid collection device is by centrifugation.

The retrieved cannabinoid in the removed extraction buffer is suitable for direct use in an enzyme immunoassay testing for the presence or absence of the cannabinoid as further detailed herein.

IV. Enzyme Immunoassays

Using the methods and kits of the present invention, the retrieved cannabinoid is stable and suitable for direct use in an enzyme immunoassay testing for the presence or absence of the cannabinoid. “Direct use” herein means that the removed extraction buffer comprising the retrieved cannabinoid, can be directly used in an enzyme immunoassay to determine presence, absence or amount of the cannabinoid. Usually, an aliquot of the removed buffer is directly analyzed, i.e., without any additional sample preparation other than adding the assay components and/or reagents of the enzyme immunoassay.

The presence, absence or amount of the cannabinoid can be determined using a variety of assays, including, but not limited to ELISA and homogeneous enzyme immunoassays.

### A. ELISA

The present invention provides methods and kits for direct use of the removed extraction buffer comprising the retrieved cannabinoid in an enzyme immunoassay testing for the presence or absence of the cannabinoid. In a preferred embodiment of the present invention, the enzyme immunoassay is an ELISA, Enzyme-Linked Immunosorbent Assay. ELISAs are known in the art. Antibodies reactive to cannabinoids are described herein and in U.S. Pat. No. 5,817,766, the disclosure of which is incorporated herein by reference in its entirety.

### B. Homogeneous Enzyme Immunoassay

The present invention provides methods and kits for direct use of the removed extraction buffer comprising the retrieved cannabinoid in an enzyme immunoassay testing for the presence, absence or amount of the cannabinoid. In a preferred embodiment of the present invention, the enzyme immunoassay is a homogeneous enzyme immunoassay. In an even more preferred embodiment of the present invention, the homogeneous enzyme immunoassay comprises glucose-6-phosphate dehydrogenase (G6PDH).

Any sample which is reasonably suspected of containing a cannabinoid can be analyzed by the methods of the present invention. Although the homogeneous enzyme immunoassays of this invention are useful to identify cannabinoids in any bodily fluid, this invention is particularly useful to identify and determine the amount of a cannabinoid in an oral fluid sample. Thus, in a preferred embodiment, an oral fluid sample suspected of containing a cannabinoid is contacted with an enzyme-cannabinoid conjugate, preferably a G6PDH-cannabinoid conjugate, an, antibody or receptor reactive to the cannabinoid, a substrate for the enzyme (e.g., glucose-6-phosphate and NAD⁺ or NADP⁺ for G6PDH) and a homogeneous competitive enzyme immunoassay is carried out as described herein.

In general, this assay works as follows (See also U.S. patent application Ser. No. 10/927,823, the disclosure of which is incorporated herein in its entirety): G6PDH, is provided and its starting specific activity is determined (by measuring the NADH or NADPH produced by G6PDH) or provided by a commercial supplier of G6PDH. G6PDH converts nicotinamide adenine dinucleotide (NAD⁺) or nicotinamide adenine dinucleotide phosphate (NADP⁺) to NADH or NADPH, respectively, resulting in an absorbance change that can be measured spectrophotometrically at 340 nm. “Absorbance” means a signal measured in a spectrophotometer or similar device. Usually, the signal is given as milli-absorbant units/minute (mA/min; see Examples 1-6).

Next, the G6PDH is covalently linked to a cannabinoid, resulting in an G6PDH-cannabinoid conjugate. The enzymatic activity of G6PDH of the G6PDH-cannabinoid conjugate is decreased due to covalent linkage of cannabinoid. This decrease in enzymatic activity is referred to as ‘deactivation.’ Next, an antibody or a receptor reactive to the cannabinoid binds to the cannabinoid of the G6PDH-cannabinoid conjugate. Binding of the antibody or receptor leads to an additional decrease of G6PDH activity. This additional decrease is referred to as ‘inhibition,’ to distinguish it from the deactivation. Upon the addition of a sample containing the same cannabinoid linked to G6PDH, some of the antibodies or receptors bound to the G6PDH-cannabinoid conjugate now bind to the free cannabinoid in the sample and release the G6PDH-cannabinoid conjugate leading to an increase in G6PDH activity. This increase is referred to as ‘reversible inhibition.’ Once calibrated, as described herein, the cannabinoid concentration in the sample is measured in terms of increased G6PDH enzyme activity. Thus, the assay is based on competition between the G6PDH-cannabinoid conjugate and the free cannabinoid in the sample for a fixed amount of specific antibody(ies) or receptor(s).

In the absence of cannabinoid(s) in the sample, the specific antibody(ies) or receptors remain bound to the G6PDH-cannabinoid conjugate causing no change in enzyme activity. On the other hand, when cannabinoid(s) is(are) present in the sample, antibody(ies) or receptors would bind to the free cannabinoid(s) in the sample and the enzymatic activity of the now unbound G6PDH-cannabinoid conjugate is increased (‘reversible inhibition’). Thus, the activity of G6PDH depends upon the concentration of the cannabinoid in the sample. The greater the cannabinoid concentration in a sample, such as oral fluid, the greater the activity of G6PDH. Enzymatic activity is determined by measuring the formation of reduced nicotineamide adenine dinucleotide (NADH) at 340 nm. Thus, a change in absorption, measured in absorbent or milli-absorbant units, can be correlated to cannabinoid concentration in a given sample.

### Measuring NADH—Autoanalyzer

The enzymatic activity of, for example, G6PDH can be measured by quantitative, semi-quantitative and qualitative methods. G6PDH enzymatic activity is determined by adding glucose-6-phosphate and NAD⁺ or NADP⁺ to the assay medium and detecting either the disappearance of one of these substrates or the appearance of NADH, NADPH, or D-glucono-δ-lactone-6-phosphate. Typically, the production of NADH or NADPH per unit time (usually in minutes) is measured using a spectrophotometer.

Measuring the signal produced by the methods of this invention can be applied easily to automated analyzers for laboratory, clinical, or high-throughput analysis. Examples of automated laboratory analyzers are COBAS INTEGRA and ROCHE/HITACHI series analyzers (Roche Diagnostics, Indianapolis, Ind.) and Olympus series (Texas).

The signal producing system may also include G6PDH and a chromophoric substrate, where the chromophoric substrate is enzymatically converted to dyes which absorb light in the ultraviolet or visible region. Phosphors or fluorescers substrate are also contemplated by this invention.

Other detection methods will be apparent to those skilled in the art. By appropriate choice of components for producing a detectible signal, the detectible signal nay be observed visually or by means of various apparatus, i.e., detection means, such as spectrophotometers, fluorometers, scintillation counters, etc.

Various techniques and combinations of reagents may be employed to enhance the production of the detectible signal.

The following will describe the individual components and parameters of the homogenous enzyme immunoassay in greater detail (See also U.S. patent application Ser. No. 10/927,823, the disclosure of which is incorporated herein in its entirety).

### Glucose-6-Phoshate Dehydrogenase (G6PDH)

The invention provides an homogeneous enzyme assay system comprising an enzyme-cannabinoid conjugate, comprising an enzyme and a cannabinoid.

In a preferred embodiment of the invention the enzyme is glucose-6-phosphate dehydrogenase (G6PDH). Included within G6PDH are proteins and polypeptides that are functionally defined by converting glucose-6-phosphate and NAD (or NADP) to 6-P-glucuronate and NADH (or NADPH). Those of skill in the art recognize that G6PDH proteins or G6PDH polypeptides can be modified in a variety of ways including the addition, deletion and substitution of amino acids.

G6PDH may be capable of using both NADP⁺ and NAD⁺, such as those isolated from Leuconostoc mesenteroides, A. suboxydans, P. aeruginosa, Pseudomonas W6, H. eutropha H-16, Hydrogenomonas facilis, Arthrobacter 7C, A. beijerickii, T. ferrooxidans, B. licheniforinis, P. denitrificans, C. crescentus, L. lactis, and R. spheroides. Alternatively, G6PDH may be capable of using NAD⁺ as a preferred cofactor such as those isolated from P. fluorescens and one of the G6PDHs from P. multivorans, or may be NAD⁺ specific such as one of the G6PDHs from A. xylinum.

In a preferred embodiment of the present invention, G6PDH is from a natural source, i.e., is a naturally occurring G6PDH, such as from yeast, bacteria, or fungi. In another embodiment of the invention, the G6PDH is a recombinant G6PDH. In yet another embodiment of the invention, the G6PDH is a mutated G6PDH. Thus, G6PDHs differing from any naturally occurring G6PDH may be generated by using molecular DNA cloning technologies as known in the art. G6PDHs with amino acid substitutions, deletions, or insertions, or any combination thereof may be generated (see U.S. Pat. No. 6,033,890) and used in the methods of this invention.

G6PDH from various sources are also commercially available, e.g., from Sigma, Biochemica, Boehringer Mannheim, USB Biochemical, and OYC International Inc.

### G6PDH Substrates

The enzymatic activities of G6PDH, the G6PDH-cannabinoid conjugate, the G6PDH-cannabinoid conjugate with bound antibody and the G6PDH-cannabinoid conjugate with bound antibody competing for cannabinoid binding in a test sample are determined. Determination of enzymatic activity is dependent on a substrate and co-enzyme for G6PDH. A suitable substrate for G6PDH is glucose-6-phosphate (G6P). Suitable co-enzymes or cofactors for G6PDH are NAD (NAD⁺) and NADP (NADP⁺). G6PDH converts G6P and co-enzymes into 6-P-glucuronate and NADH and NADPH, respectively. Thus, generally, in order to measure G6PDH activity, G6P and NAD⁺ or NADP⁺ are added to the assay. Cofactor analogs, such as thio-NAD⁺, thio-NADH, thio-NADP⁺, or thio-NADPH may also be used.

Typically, substrate and co-enzyme or co-factors for G6PDH are not labeled and the signal generated by G6PDH, i.e., the amount of NADPH or NADH, is measured in a spectrophotometer as described herein. Alternatively, the substrate and or co-enzymes may be labeled and the signal generated by G6PDH may be detected by other means, depending on the label, such as a fluorometer or scintillation counter, or the like.

### Other Enzymes Suitable for Use in the Present Invention

The invention provides an enzyme-cannabinoid conjugate, comprising an enzyme and a cannabinoid. In a preferred embodiment of the invention, the enzyme is G6PDH. In another embodiment of the invention, the enzyme is an enzyme other G6PDH. Additional enzymes that are useful for the present invention and which use NAD (NAD⁺) as a co-enzyme and generate NADH include alcohol dehydrogenase, glutamic dehydrogenase, malic dehydrogenase, isocitric dehydrogenase, α-glycerol phosphate dehydrogenase, lactic dehydrogenase, and glyceraldehydes-3-phosphate dehydrogenase. Additional enzymes that are useful for the present invention and which use NADP (NADP⁺) as a co-enzyme and generate NADPH include gluthathione reductase, quinine reductase, nitrate reductase, and glutamic dehydrogenase. In addition, a large number of enzymes and co-enzymes useful in the methods of the present invention are disclosed in U.S. Pat. Nos. 4,275,149 and 4,318,980, which are incorporated herein by reference. Employing one or more of the above enzymes may further increase the sensitivity of the present immunoassay.

### Conjugation

When the methods and kits of the invention use a competition immunoassay, such as a homogeneous immunoassay as described herein, an enzyme-cannabinoid conjugate comprising an enzyme covalently linked or conjugated to a cannabinoid is provided. For example, G6PDH is conjugated to a cannabinoid resulting in a G6PDH-cannabinoid conjugate. “Conjugation” is any process wherein two subunits are linked together to form a conjugate, in particular and within the context of the present invention, an enzyme-cannabinoid conjugate. An “enzyme-cannabinoid conjugate” refers to a covalent fusion or covalent linkage between an enzyme of interest, such as glucose-6-phosphate dehydrogenase and a cannabinoid.

Conjugation can be achieved via conventional chemical reactions as known in the art. Among them, the simplest reaction to coupling a cannabinoid (or a hapten) to G6PDH is through the formation of a peptide bond (—CONH₂). For example, using a carboxyl (—COOH) group on a cannabinoid (or a hapten) to react with an amino group (—NH₂) on the G6PDH enzyme (Biochem. and Biophys. Res. Comm., (1989) vol. 160:3, 1290-1295).

Some cannabinoids are capable of binding directly to G6PDH. Others are not capable of covalent binding directly. Such cannabinoids are rendered capable of covalently binding to G6PDH by the addition of a linking group (i.e, definition of haptens) that can covalently bind to a group on G6PDH (for instance, to an amino, hydroxyl, carboxyl or mercapto group). “Linking group” refers to a portion of a structure which connects 2 or more substructures. A linking group has at least one uninterrupted chain of atoms extending between the substructures. Such linking groups may comprise, for instance, amino acids having one or more free amino or free hydroxyl groups, or may comprise carbonyl, thiocarbonyl, or carboxyl groups, or compounds containing such groups. Linking groups commonly used for this purpose include N-hydroxysuccinimide and other succinimide or maleimide-containing moieties, and 1-(3-dimethylpropyl)-3-ethylcarbodiimide. A detailed discussion of such linking groups is found in U.S. Pat. No. 3,817,837 and in U.S. patent application Ser. No. 10/927,823, which are incorporated by reference in their entirety.

After the conjugation, the enzyme-cannabinoid conjugate may be purified. Suitable purification procedures are known in the art and include dialysis against aqueous/organic and aqueous solutions such as water/DMF or water, or by gel filtration chromatography on supports such as Sephadex, and the like.

### Cannabinoid Antibodies

The present methods and kits of the invention provide antibodies binding to the cannabinoid of the enzyme-cannabinoid conjugate. “Antibody” refers to a protein functionally defined as a binding protein (a molecule able to bind to a specific epitope on an antigen) and structurally defined as comprising an amino acid sequence that is recognized by one of skill as being derived from the framework region of an immunoglobulin encoding gene. It includes whole antibody, functional fragments, modifications or derivatives of the antibody. It can also be a genetically manipulated product, or chimeric antibody, such as a humanized antibody. Antibodies can be a polyclonal mixture or monoclonal. Antibodies can be intact immunoglobulins derived from natural sources or from recombinant sources and can be immunoreactive portions of intact immunoglobulins. Antibodies may exist in a variety of forms including, for example, Fv, Fab, and F(ab)₂, as well as in single chains. Single-chain antibodies, in which genes for a heavy chain and a light chain are combined into a single coding sequence, may also be used.

Antibodies contemplated by this invention include one or more polypeptides substantially encoded by immunoglobulin genes or fragments of immunoglobulin genes. Immunoglobulin genes include the kappa, lambda, alpha, gamma, delta, epsilon and mu constant region genes, as well as myriad immunoglobulin variable region genes. Immunoglobulin light chains are classified as either kappa or lambda. Immunoglobulin heavy chains are classified as gamma, mu, alpha, delta, or epsilon, which define immunoglobulin classes, IgG, IgM, IgA, IgD and IgE, respectively.

The term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer, which may be expressed from a nucleic acid including V_{H}- and V_{L}-encoding sequences either joined directly or joined by a peptide-encoding linker (Huston, et al. (1988) Proc. Natl. Acad. Sci. USA, 85: 5879-5883). While the V_{H} and V_{L} are connected to each as a single polypeptide chain, the V_{H} and V_{L} domains associate non-covalently.

Particularly preferred antibodies include di-antibodies, mini-antibodies, humanized antibodies, or chimeric antibodies.

Usually, an antibody reactive to the cannabinoid has an affinity for the cannabinoid (Ka) in the range of at least 1×10⁸ M⁻¹ to at least 5×10⁸ M⁻¹, more preferably in the range of at least 5×10⁸ M⁻¹ to at least 2×10⁹M⁻¹, most preferably in the range of at least 2×10⁹ M⁻¹ to at least 5×10⁹ M⁻¹. In a preferred embodiment of this invention, the Ka is ≧×10¹⁰ M⁻¹. In another preferred embodiment of this invention, the Ka is >1×10¹¹ M⁻¹. “Antibody reactive to a cannabinoid” means that the antibody has an area on its surface or in a cavity which specifically binds to a particular cannabinoid, i.e., it has a binding affinity (usually expressed as Ka) for the cannabinoid.

Antibodies can be prepared by techniques that are well known in the art. Polyclonal antibodies can be prepared by injecting an antigen, such as a cannabinoid, into a wide variety of vertebrates in accordance with conventional methods for the production of antibodies. Likewise, monoclonal antibodies useful in this invention may be produced according to hybrid cell line technologies. Antibodies specific to cannabinoids can be obtained from commercial sources, such as Cortex Biochem, Inc., Biodesign International, and Fitzgerald Industry, Inc.

### Cannabinoid Receptors

The methods and kits of the invention provide a receptor that is reactive to a cannabinoid. Within the present invention, “receptor” refers to any compound or composition capable of recognizing a particular spatial and polar organization of a cannabinoid, i.e., an epitope or determinant site on a cannabinoid. A “receptor reactive to a cannabinoid” means that the receptor has an area on its surface or in a cavity which specifically binds to a particular cannabinoid, i.e., it has a binding affinity (usually expressed as Ka) for the cannabinoid.

Two cannabinoid receptors have been described, the CB1 receptor expressed primarily in the brain (Matsuda et al., Nature (1990) 346:561-564), and CB2 receptors expressed by cells of the immune system (Munro et al., Nature (1993) 365:61-65). Thus, cannabinoids are ligands for CB1 and CB2 receptors (“Ligand” refers to any organic compound for which a receptor naturally exists or can be prepared).

The enzyme-cannabinoid conjugate, more specifically, the G6PDH-cannabinoid conjugate, can be mixed with a CB1 or CB2 receptor that is specifically reactive to the G6PDH-cannabinoid conjugate and the free cannabinoid. The CB1 or CB2 receptor can be any composition that can bind effectively and specifically to a cannabinoid and when bound to an enzyme-cannabinoid conjugate cause an inhibition of the activity of the enzyme, such as G6PDH. Suitable CB1 or CB2 receptors would include, but are not limited to, soluble forms of natural CB1 or CB2 receptors, recombinant CB1 or CB2 receptors and derivatives and fragments that are reactive to a cannabinoid.

### Calibrators and References

The retrieved cannabinoid in the removed extraction buffer is suitable for direct use in an enzyme immunoassay testing for the presence or absence of the cannabinoid.

When enzyme immunoassays are used to determine the absence, presence, or amount of a cannabinoid, the present invention provides formulation of calibrators, controls or references to validate enzyme immunoassay performance and to determine the amount of cannabinoid in an oral fluid sample suspected of containing a cannabinoid. “Calibrator” or “Reference” refers to any standard or reference material containing a known amount of the cannabinoid to be measured.

The formulation of calibrators, controls and references include buffers that comprise tris buffer, protein, sodium chloride, non-ionic detergent, or sodium azide. Usually, the buffer capacity of the formulation buffer for the calibration compound is in the range of from about 50 to about 200 mM, preferably in the range of from about 75 to about 150 mM, more preferably in the range of from about 80 to about 100 mM.

Likewise, known amounts of cannabinoid may also be added to an oral fluid sample that is clearly negative for the cannabinoid to be measured.

By comparing, for example, the observed G6PDH enzymatic activity obtained by analyzing an oral fluid sample with the G6PDH enzymatic activity obtained from an immunoassay having a known amount of cannabinoid, one can qualitatively and quantitatively determine the cannabinoid of interest in the oral fluid sample analyzed.

Thus, in one embodiment of the invention, calibration components (or calibrators) are provided. The calibration component contains a known amount of the cannabinoid to be determined. For example, the calibration component may comprise cannabinoid samples containing 0, 5, 10, 20, 50, 100, and 250 ng/ml of a cannabinoid.

A sample suspected of containing a cannabinoid of interest and the calibration component (or calibrator) containing a known amount of the same cannabinoid are assayed under similar conditions (i.e., similar buffer and sample volumes, etc.). Analyzing the known cannabinoid samples results in a standard calibration curve. The cannabinoid concentration in the sample suspected of containing a cannabinoid of interest is then calculated by comparing the results obtained for the unknown specimen with the results obtained for the standard.

VI. Kits for Retrieving a Cannabinoid from an Oral Fluid Collection Device

The invention further provides for kits for retrieving a cannabinoid from an oral fluid collection device having adsorbed the cannabinoid, the kit comprising (i) an oral fluid collection device; (ii) an extraction buffer comprising β-cyclodextrin or DMF; and (iii) instructions for retrieving the cannabinoid from the oral fluid collection device.

In one embodiment of the present invention, the kit includes an extraction buffer comprising from about 1% (w/v) to about 2% (w/v) β-cyclodextrin. In another embodiment, the extraction buffer comprises from about 20% (w/v) to about 35% (w/v) DMF. In a preferred embodiment, the kit comprises an extraction buffer comprising a mixture of from about 20% (w/v) to about 35% (w/v) DMF and from about 0.5% (w/v) to about 5% (w/v) β-cyclodextrin.

In a preferred embodiment of the present invention, the kit comprises a β-cyclodextrin that is a modified β-cyclodextrin selected from the group consisting of hydroxypropyl-β-cyclodextrin and sulfobutyl-ether-β-cyclodextrin. In another embodiment, the extraction buffer comprises a mixture of hydroxypropyl-β-cyclodextrin and sulfobutyl-ether-β-cyclodextrin. In a preferred embodiment of the present invention, the extraction buffer comprises a mixture of hydroxypropyl-β-cyclodextrin and β-cyclodextrin or a mixture of sulfobutyl-ether-β-cyclodextrin and β-cyclodextrin. In another embodiment of the present invention, the extraction buffer comprises a mixture of hydroxypropyl-β-cyclodextrin, sulfobutyl-ether-β-cyclodextrin and β-cyclodextrin.

In one embodiment of the present invention, the kit comprises an extraction buffer from about 50 mM to about 100 mM Tris and a pH range of from about 7.2 to about 8.2.

In a preferred embodiment of the present invention, the cannabinoid retrieved from the oral fluid collection device is Δ⁹-tetrahydrocaninabinol.

In a preferred embodiment of the present invention, the kit further comprises reagents for performing an enzyme immunoassay to determine the presence or absence of the cannabinoid. The enzyme immunoassay can be a homogeneous enzyme immunoassay or an ELISA. In another preferred embodiment, the homogeneous enzyme immunoassay comprises glucose-6-phosphate dehydrogenase.

Reagents for performing an enzyme immunoassay may comprise one or more of the following components as fully described herein: (a) an enzyme-cannabinoid conjugate comprising glucose-6-phosphatase dehydrogenase (G6PDH) covalently linked to a cannabinoid, (b) an antibody or receptor reactive to the cannabinoid, (c) an enzyme substrate for G6PDH, (d) a co-enzyme for G6PDH, (e) an assay buffer, (f) calibrators, controls or references and the like, and (g) an instruction manual describing how to perform the homogenous enzyme immunoassay.

In one embodiment of the invention, reagents and compositions useful in the methods of the invention, are provided in a packaged combination. The reagents or compositions may be in the same or in separate containers depending on cross-reactivity and/or stability of the reagents or compositions. The reagents or compositions may be in liquid or in lyophilized form. Where reagents or compositions are provided as dry powders, i.e., usually lyophilized, excipients or buffers are included, so that upon dissolution, the reagent solutions will have the appropriate concentrations for performing the methods of this invention.

In one embodiment of the invention, the kit includes two or more different G6PDH-cannabinoid conjugates. These two or more G6PDH-cannabinoid substitutes can be used to determine the amount of two or more cannabinoids in an oral fluid sample either subsequently or simultaneously as described in U.S. patent application Ser. No. 10/163,018 (Publication No. US-2003-0224373-A1), hereby incorporated in its entirety.

From the foregoing it is believed that those familiar with the art will readily recognize and appreciate the novel concepts and features of the present invention. Numerous variations, changes and substitutions of equivalents will present themselves from persons skilled in the art and may be made without necessarily departing from the scope and principles of this invention. As a result, the embodiments described herein are subject to various modifications, changes and the like, with the scope of this invention being determined solely by reference to the claims appended hereto.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

The invention is further illustrated by the following examples, which are only illustrative and are not intended to limit the definition of the invention in any way.

### EXAMPLES

### Example 1

### Δ⁹-THC Sticks to Solid Surfaces

The following experiment demonstrates the problem of cannabinoids, especially, Δ⁹-THC to adhere to surfaces of collection devices.

Artificial oral fluid samples (Sciteck) were spiked with Δ⁹-THC at 25 ng/ml and 100 ng/ml concentrations. Two (2) ml of the spiked oral fluid was adsorbed to an oral fluid collection device (cotton swab; obtained from Salivette, provided by Sarstedt, Inc., N.C.). The oral fluid cotton swab was stored in a refrigerator overnight.

The oral fluid sample was removed from the cotton swab by centrifugation and assayed using the THC oral fluid assay reagents on a Hitachi 717 instrument. The result of this glucose-6-phosphate dehydrogenase assay is shown in the following table:

| Δ⁹-THC Concentration | Reference | Recovered from swab |
|---|---|---|
| 0 ng/ml (negative) | 320 mA/min | 319 mA/min |
| 25 ng/ml | 350 mA/min | 321 mA/min |
| 100 ng/ml | 420 mA/min | 326 mA/min |

Reference corresponds to spiked sample without adsorption to collection device. The result clearly indicates that Δ⁹-THC was effectively adsorbed from the oral fluid to the collection device (here a cotton swab) and could not be efficiently retrieved from the collection device to allow accurate measuring using the G6PDH homogeneous enzyme immunoassay.

### Example 2

### Selection of Organic Solvents Not Interfering with G6PDH Homogeneous Enzyme Immunoassay

The following experiment is performed to identify organic solvents in an extraction buffer for retrieving a cannabinoid that do not significantly interfere with enzyme-based detection assays, especially not with a homogeneous enzyme immunoassay employing G6PDH.

A negative buffer (50 mM Tris, pH 7.2) was mixed with 15% (w/v) of DMF, 30% (w/v) DMF, 30% (w/v) DMSO, 30% (w/v) ethanol, and 30% (w/v) dioxane), respectively. The solutions were well mixed and stored in a refrigerator overnight prior to use. The extraction buffers were used as negative samples and assayed using the THC oral fluid assay reagents on a Hitachi 717 instrument. The result of this glucose-6-phosphate dehydrogenase assay is shown in the following table:

| Buffer with | Rate (mA/min) |
|---|---|
| No solvent | 320 |
| 15% (w/v) DMF | 328 |
| 30% (w/v) DMF | 327 |
| 30% (w/v) DMSO | 312 |
| 30% (w/v) ethanol | 314 |
| 30% (w/v) dioxane | 303 |

The result indicates that the extraction buffer/organic solvent combinations tested are compatible with and do not interfere significantly with the G6PDH homogeneous enzyme immunoassay.

### Example 3

### Retrieving Δ⁹-THC from an Oral Fluid Collection Device Using Extraction Buffers Comprising an Organic Solvent

The following experiment is performed to demonstrate that certain organic solvents can retrieve a cannabinoid, especially, Δ⁹-THC, from a collection device having adsorbed the cannabinoid.

Artificial oral fluid (Sciteck) was spiked with various concentrations of Δ⁹-THC and adsorbed to a collection device (cotton swab) as described in Example 1. After centrifugation to remove the oral fluid sample, the semi-dry cotton swabs were treated with extraction buffers comprising the organic solvents as indicated below. The extraction buffer volume was equal to the volume of oral fluid spiked with Δ⁹-THC. After approximately 6 minutes, the extraction buffer was compressed out of the oral fluid collection device and into a Hitachi 717 instrument sample cup and assayed using the THC oral fluid assay reagents. The results were recorded as mA/min.

| Buffer + Organic | Recovery | | |
|---|---|---|---|
| Solvent | 0 ng/mL THC | 25 ng/ml THC | 100 ng/ml THC |
| 15% (w/v) DMF | 320 | 326 | 325 |
| 30% (w/v) DMF | 321 | 342 | 410 |
| 30% (w/v) DMSO | 312 | 331 | 338 |
| 30% (w/v) ethanol | 314 | 323 | 324 |
| 30% (w/v) dioxane) | 303 | 315 | 330 |
| no organic solvent | 319 | 321 | 326 |

The results indicate that all buffers comprising the organic solvent indicated are suitable for retrieving Δ⁹-THC from an oral fluid collection device. In this example, 30% (w/v) DMF worked best, retrieving >95% of the adsorbed Δ⁹-THC. 15% (w/v) DMF, 30% (w/v) of DMSO, and 30% (w/v) dioxane, each retrieved <10% of the adsorbed Δ⁹-THC. 30% (w/v) ethanol retrieved <5% of the adsorbed Δ⁹-THC. Compare to Example 1, where the reference for 100 ng/ml Δ⁹-THC resulted in 420 mA/min.

### Example 4

### Retrieving Δ⁹-THC from an Oral Fluid Collection Device Using Extraction Buffers Comprising 30% (w/v) DMF

The following experiment is performed to demonstrate that an extraction buffer comprising 30% DMF can retrieve a cannabinoid, especially, Δ⁹-THC, from a collection device having adsorbed the cannabinoid.

Spiked oral fluid samples with various Δ⁹-THC concentrations were adsorbed to an oral fluid collection device (cotton swab) and stored in a refrigerator overnight. The samples were removed from the cotton swab by centrifugation and assayed using the THC oral fluid assay reagents on a Hitachi 717 instrument. The semi-dry oral fluid collection devices were then incubated with an extraction buffer comprising 30% (w/v) DMF at room temperature for approximately 5 minutes. The extraction buffers were compressed out of the cotton swab into Hitachi 717 sample cups and assayed using the THC oral fluid assay reagents. The results are presented as mA/min.

| | Recovery | | |
|---|---|---|---|
| Spiked | | | 30% |
| Δ⁹-THC | Reference | From Swab | (w/v) DMF |
| 0 ng/ml | 312 | 310 | 314 |
| 25 ng/ml | 363 | 308 | 360 |
| 50 ng/ml | 400 | 310 | 384 |
| 100 ng · ml | 428 | 311 | 413 |
| 200 ng/ml | 430 | 312 | 417 |

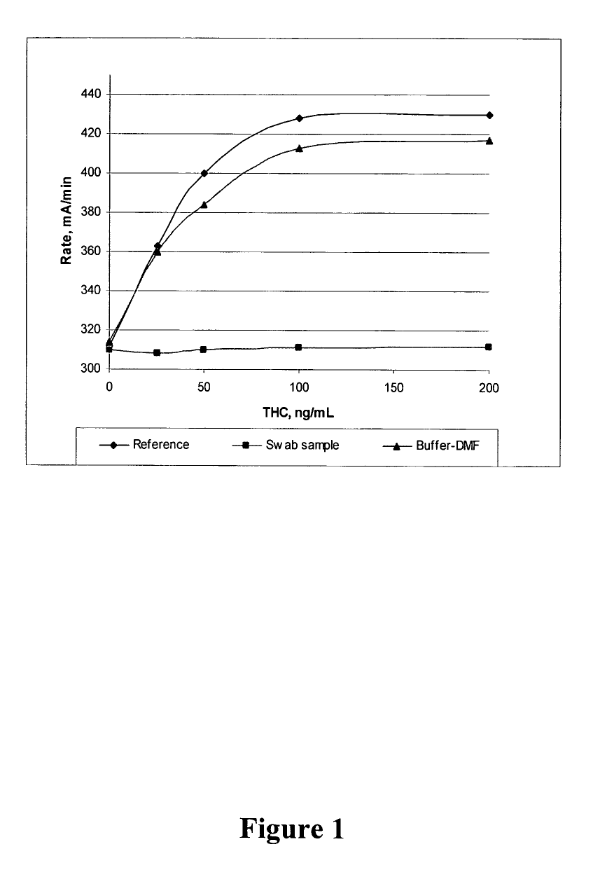

Reference corresponds to spiked sample without adsorption to collection device. The result shows that Δ⁹-THC is efficiently adsorbed to the cotton swab, as there is no recovery in the sample recovered from the swab. However, the extraction buffer comprising 30% (w/v) DMF is suitable to recover a significant amount of the adsorbed Δ⁹-THC. The result of this experiment is shown in FIG. 1.

### Example 5

### Retrieving Δ⁹-THC from an Oral Fluid Collection Device Using Extraction Buffers Comprising Various Concentrations of DMF

The following experiment is performed to determine the concentration of DMF in an extraction buffer that can efficiently retrieve a cannabinoid, especially, Δ⁹-THC, from a collection device having adsorbed the cannabinoid.

Artificial oral fluid (Sciteck) was spiked with Δ⁹-THC at 25, 50, and 100 ng/ml. The spiked samples (1 ml) were adsorbed to an oral fluid collection device (cotton swab) and stored in a refrigerator overnight. The samples were removed from the cotton swab by centrifugation and assayed using the THC oral fluid assay reagents on a Hitachi 717 instrument. The results indicated that >95% of Δ⁹-THC was depleted from the sample, i.e., was adsorbed to the cotton swab. One ml of extraction buffers each comprising a different concentration of DMF was then added to the semi-dry cotton swabs, which were then incubated at room temperature. After 10 minutes the extraction buffers were compressed out of the cotton swab into Hitachi 717 sample cups and assayed using the THC oral fluid assay reagents. The results are presented as mA/min.

| | Recovery | | | |
|---|---|---|---|---|
| | | 25% | 30% | 35% |
| Spiked Δ⁹-THC | Reference | (w/v) DMF | (w/v) DMF | (w/v) DMF |
| 0 ng/ml | 312 | 318 | 320 | 319 |
| 25 ng/ml | 350 | 345 | 344 | 343 |
| 50 ng/ml | 382 | 361 | 367 | 372 |
| 100 ng/ml | 416 | 383 | 391 | 399 |

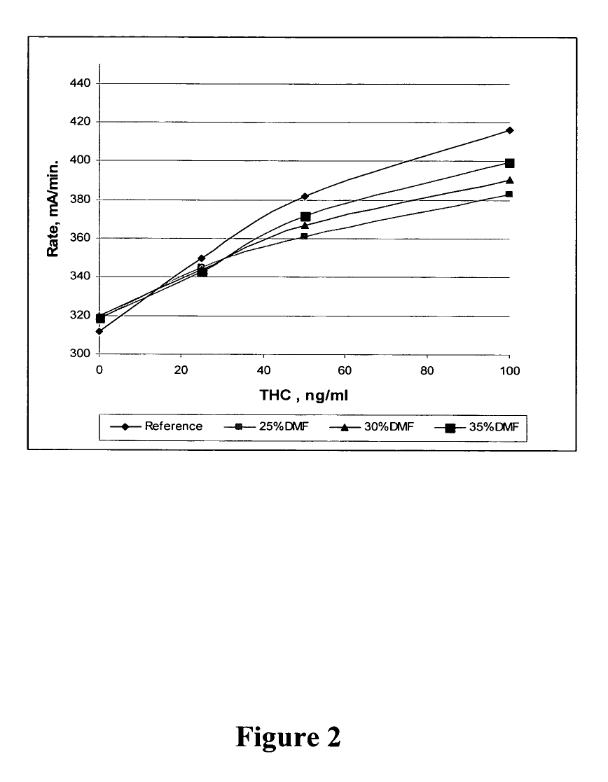

Reference corresponds to spiked sample without adsorption to collection device. The result shows that extraction buffers comprising 25% (w/v) to 35% (w/v) DMF are suitable to recover a significant amount of the adsorbed Δ⁹-THC. The result of this experiment is shown in FIG. 2.

### Example 6

### Retrieving Δ⁹-THC from an Oral Fluid Collection Device Using Extraction Buffers Comprising DMF and Various O—Cyclodextrins

The following experiment is performed to determine whether an extraction buffer comprising DMF and various modified β-cyclodextrins can efficiently retrieve a cannabinoid, especially, Δ⁹-THC, from a collection device having adsorbed the cannabinoid.

Four different extraction buffers were prepared. In this example, the extraction buffer is 100 mM Tris and 0.9% sodium chloride, pH 8.0, and further comprising:

1% (w/v) hydroxypropyl-β-cyclodextrin;

1% (w/v) hydroxypropyl-β-cyclodextrin and 30% (w/v) DMF; or

1% (w/v) sulfobutyl-ether-β-cyclodextrin; or

1% (w/v) sulfobutyl-ether-β-cyclodextrin and 25% (w/v) DMF.

Artificial oral fluid (Sciteck) was spiked with Δ⁹-THC at 25, 50, and 100 ng/ml. The spiked oral fluid samples (2.4 ml) were adsorbed to an oral fluid collection device (cotton swab) and stored in a refrigerator overnight. Part of the oral fluid samples were compressed out of the cotton swab and assayed. It was found that >95% of Δ⁹-THC was adsorbed to the cotton swab. The rest of the oral fluid samples were removed from the cotton swab by centrifugation. 2.4 ml of extraction buffers indicated above were then added to the semi-dry cotton swabs. The soaked swaps were then incubated with the extraction buffers at room temperature. After 15 minutes part of the extraction buffers were compressed out of the cotton swab into Hitachi 717 sample cups and assayed using the THC oral fluid assay reagents. The rest of the extraction buffers was centrifuged into plastic containers and assayed. The results are presented as mA/min.

| | | 1% hydroxypropyl-β-cyclodextrin | | | |
|---|---|---|---|---|---|
| | | No DMF | | 30% (w/v) DMF | |
| Spiked Δ⁹-THC | Reference | compressed | centrifuged | compressed | centrifuged |
| 0 ng/ml | 300 | 319 | 316 | 338 | 330 |
| 25 ng/ml | 340 | 344 | 330 | 385 | 340 |
| 50 ng/ml | 400 | 400 | 335 | 412 | 360 |
| 100 ng/ml | 420 | 435 | 373 | 421 | 398 |
| | | 1% sulfobutyl-ether-β-cyclodextrin | | | |
| | | No DMF | | 25% (w/v) DMF | |
| Spiked Δ⁹-THC | Reference | compressed | centrifuged | compressed | centrifuged |
| 0 ng/ml | 300 | 310 | 308 | 322 | 323 |
| 25 ng/ml | 340 | 358 | 334 | 363 | 345 |
| 50 ng/ml | 400 | 418 | 366 | 404 | 350 |
| 100 ng/ml | 420 | 435 | 391 | 418 | 385 |

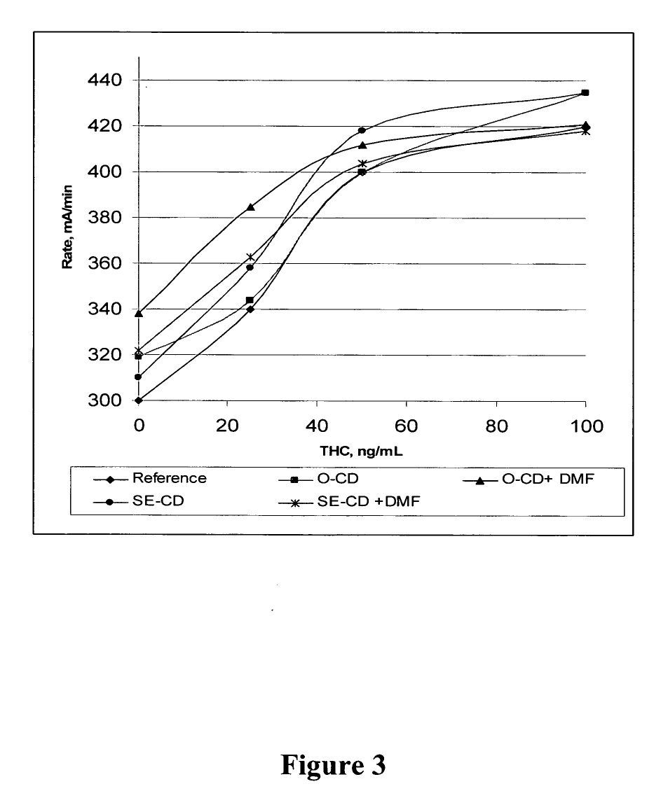

The results show that all four extraction buffers tested are suitable to retrieve Δ⁹-THC from an oral fluid collection device having adsorbed the Δ⁹-THC. 1% (w/v) of sulfobutyl-ether-β-cyclodextrin works very efficiently. The results of this experiment are shown in FIG. 3.

What is claimed is:

1. A method of retrieving a cannabinoid from an oral fluid collection device having adsorbed the cannabinoid, the method comprising the steps of:
(a) removing the oral fluid from the oral fluid collection device;
(b) adding an extraction buffer comprising β-cyclodextrin or N,N-dimethylformamide (DMF) to the oral fluid collection device; and
(c) removing the extraction buffer from the oral fluid collection device;
wherein the retrieved cannabinoid in the removed extraction buffer is suitable for direct use in an enzyme immunoassay testing for the presence or absence of the cannabinoid.

2. A method according to claim 1, further comprising the step of:
(d) using the oral fluid collection device to collect the oral fluid from an individual suspected of having consumed the cannabinoid.

3. A method according to claim 1, wherein the extraction buffer comprises from about 1% (w/v) to about 2% (w/v) β-cyclodextrin.

4. A method according to claim 1, wherein the extraction buffer comprises from about 20% (w/v) to about 35% (w/v) DMF.

5. A method according to claim 1, wherein the extraction buffer comprises a mixture of from about 20% (w/v) to about 35% (w/v) DMF and from about 0.5% (w/v) to about 5% (w/v) β-cyclodextrin.

6. A method according to claim 1, wherein the β-cyclodextrin is a modified β-cyclodextrin selected from the group consisting of hydroxypropyl β-cyclodextrin and sulfobutyl-ether-β-cyclodextrin.

7. A method according to claim 6, wherein the extraction buffer comprises a mixture of hydroxypropyl β-cyclodextrin and sulfobutyl-ether-β-cyclodextrin.

8. A method according to claim 1, wherein the extraction buffer further comprises hydroxypropyl β-cyclodextrin or sulfobutyl-ether-β-cyclodextrin.

9. A method according to claim 1, wherein the extraction buffer is from about 50 mM to about 100 mM Tris and has a pH range of from about 7.2 to about 8.2.

10. A method according to claim 1, wherein the cannabinoid is Δ⁹-tetrahydrocaninabinol.

11. A method according to claim 1, wherein step (a) comprises filtration or centrifugation.

12. A method according to claim 1, wherein step (c) comprises compressing the buffer out of the oral fluid collection device.

13. A method according to claim 1, wherein the enzyme immunoassay is a homogeneous enzyme immunoassay.

14. A method according to claim 13, wherein the homogeneous enzyme immunoassay comprises glucose-6-phosphate dehydrogenase.

15. A method according to claim 1, wherein the enzyme immunoassay is an ELISA.

16. A kit for retrieving a cannabinoid from an oral fluid collection device having adsorbed the cannabinoid, the kit comprising:
(i) an oral fluid collection device;
(ii) an extraction buffer comprising β-cyclodextrin or DMF; and
(iii) instructions for retrieving the cannabinoid from the oral fluid collection device.

17. A kit according to claim 16, wherein the extraction buffer comprises from about 1% (w/v) to about 2% (w/v) β-cyclodextrin.

18. A kit according to claim 16, wherein the extraction buffer comprises from about 20% (w/v) to about 35% (w/v) DMF.

19. A kit according to claim 16, wherein the extraction buffer comprises a mixture of from about 20% (w/v) to about 35% (w/v) DMF and from about 0.5% (w/v) to about 5% (w/v) β-cyclodextrin.

20. A kit according to claim 16, wherein the β-cyclodextrin is a modified β-cyclodextrin selected from the group consisting of hydroxypropyl β-cyclodextrin and sulfobutyl-ether-β-cyclodextrin.

21. A kit according to claim 20, wherein the extraction buffer comprises a mixture of hydroxypropyl β-cyclodextrin and sulfobutyl-ether-β-cyclodextrin.

22. A kit according to claim 16, wherein the extraction buffer is from about 50 mM to about 100 mM Tris and has a pH range of from about 7.2 to about 8.2.

23. A kit according to claim 16, wherein the cannabinoid is Δ⁹-tetrahydrocaninabinol.

24. A kit according to claim 16, further comprising reagents for performing an enzyme immunoassay to determine the presence or absence of the cannabinoid.

25. A kit according to claim 24, wherein the enzyme immunoassay is a homogeneous enzyme immunoassay.

26. A kit according to claim 25, wherein the homogeneous enzyme immunoassay comprises glucose-6-phosphate dehydrogenase.

27. A kit according to claim 24, wherein the enzyme immunoassay is an ELISA.
